(19) United States
(12) Patent Application Publication
(10) Pub. No.: US 2021/0386753 A1
(43) Pub. Date: Dec. 16, 2021

(54) METHODS OF ADMINISTERING INTRAVENOUS MELOXICAM PRE-OPERATIVELY AND IN COMBINATION WITH OTHER DRUGS

(71) Applicant: BAUDAX BIO, INC., Malvern, PA (US)

(72) Inventors: Randall J. MACK, Malvern, PA (US); Stewart MCCALLUM, Malvern, PA (US)

(21) Appl. No.: 17/287,489

(22) PCT Filed: Oct. 23, 2019
(86) PCT No.: PCT/US19/57692

§ 371 Date: Apr. 21, 2021

Related U.S. Application Data
(60) Provisional application No. 62/749,407, filed on Oct. 23, 2018.

(51) Int. Cl. A61K 31/5415; A61K 31/195; A61K 31/167; A61K 31/485; A61K 9/00; A61K 9/10; A61P 23/00; A61P 41/00

(52) U.S. Cl. CPC A61K 31/5415; A61K 31/195; A61K 31/167; A61P 41/00; A61K 9/0019; A61K 9/10; A61P 23/00; A61K 31/485

(57) ABSTRACT

The disclosure provides methods of treating pain in a patient who will be subjected to a surgical procedure, comprising administering meloxicam to the patient prior to start of the surgical procedure. In some embodiments, meloxicam is nanocrystalline meloxicam. The disclosure further provides methods of treating pain in a patient in need thereof, comprising administering meloxicam intravenously to the patient in combination with acetaminophen and/or gabapentin.

### CROSS-REFERENCE TO RELATED APPLICATIONS

This application claims the benefit of priority under 35 U.S.C. § 119(e) of U.S. Provisional Patent Application No. 62/749,407, filed Oct. 23, 2018, the contents of which are incorporated herein by reference in its entirety for all purposes.

### FIELD

The present disclosure relates to methods of administering meloxicam for treatment of pain, including pre-operatively and/or in combination with other drugs.

### BACKGROUND

Meloxicam (4-hydroxy-2-methyl-N-(5-methyl-2-thiazolyl)-2H-1,2-benzothiazine-3-carboxamide-1,1-dioxide) is a long-acting nonsteroidal anti-inflammatory drug (NSAID) that possesses anti-inflammatory, analgesic, and antipyretic activities, which are believed to be related to the inhibition of cyclooxygenase (COX) and subsequent reduction in prostaglandin biosynthesis. Meloxicam has been marketed by Boehringer Ingelheim Pharmaceuticals, Inc. since the 1990's as an oral agent, Mobic®. Mobic is used for treatment of symptoms of osteoarthritis and rheumatoid arthritis.

However, oral meloxicam has a slow onset of action, largely due to poor water solubility, and is not currently approved for the treatment of acute pain. The oral form has a prolonged absorption time, with the time of maximum observed plasma concentration (tₘₐₓ) being approximately 5-6 hours following oral administration, which is consistent with its poor water solubility.

Intravenous (IV) administration of the NSAID ibuprofen was approved in 2009 for pain management; however, infusion time of 30 minutes is required and it must be administered every 6 hours for treatment of pain. See CALDOLOR® Prescribing Information. In addition, patients receiving IV administration of ibuprofen, ketoroloac and other NSAIDs have suffered from relatively high rates of injection site pain or discomfort (e.g., 14%-29% reported), which prohibits faster administration times. See Gan T J, et al., Clinical Therapeutics, 2015, 37, 368-375; Zhou T J, et al. Anesth Analg. 2001; 92:1569-1575. Because current IV NSAIDs require slow injection times and are not administered pre-operatively (i.e., for preventative treatment), patients experience significant pain before onset of pain relief. Thus, there is a need for a method of administering meloxicam which can provide a faster onset of action and preventive pain treatment of acute pain (mild to moderate pain and moderate to severe pain).

Further, tens of millions of patients undergo surgical procedures every year. Many of these patients suffer from pain localized within the vicinity of the surgical site or post-surgical pain. Conventionally, NSAIDs are generally administered after the surgical procedure when the patient is already experiencing significant pain. Further, post-surgical pain treatment and management has been fraught with difficulties since drugs currently used for this purpose have a variety of prominent side effects that delay recovery, prolong hospitalization and subject certain vulnerable patient groups to the risk of serious complications. Therefore, post-surgical pain is a serious and often intractable medical problem and there is a pressing need for therapeutic interventions that can effectively treat and/or manage post-surgical pain with relatively fewer or less severe side effects.

### SUMMARY

The present disclosure provides a method of treating pain in a patient who will be subjected to a surgical procedure, comprising administering meloxicam to the patient prior to start of the surgical procedure. In some embodiments, the pain is an acute pain. In some embodiments, meloxicam is present as nanocrystalline meloxicam. In some embodiments, the nanocrystalline meloxicam is in a colloidal dispersion. In some embodiments, meloxicam is administered to the patient in an amount ranging from about 5 mg to about 180 mg or about 15 mg to about 60 mg, such as, for example 30 mg. In some embodiments, meloxicam is administered to the patient intravenously. In some embodiments meloxicam is administered to the patient intravenously over a course of about 5 seconds to about 60 seconds, about 10 second to about 60 seconds, about 15 seconds to about 30 seconds, such as, for example 15 seconds.

In some embodiments, meloxicam is administered to the patient within about 2 hours, within about 45 minutes or within about 30 minutes prior to start of the surgical procedure. In some embodiments, meloxicam is administered to the patient prior to the administration of anesthesia, while in other embodiments, meloxicam is administered to the patient after the administration of anesthesia. In some embodiments, the surgical procedure is performed on soft tissue, hard tissue or a combination thereof. In some embodiments, the surgical procedure comprises an open surgical procedure. In other embodiments, the surgical procedure comprises a laparoscopic procedure. In some embodiment, the surgical procedure comprises colorectal surgery. In other embodiments, the surgical procedure comprises unilateral total knee arthroplasty.

In some embodiments, the method of treating pain in a patient who will be subjected to a surgical procedure, comprising administering meloxicam to the patient prior to start of the surgical procedure, further comprises administering gabapentin, acetaminophen or a combination thereof to the patient prior to the start of the surgical procedure. In some embodiments, the method further comprises administering an analgesic to the patient before, during or after the surgical procedure. In some embodiments, the analgesic comprises acetaminophen, opioid, or a combination thereof; in other embodiments, the opioid is oxycodone. In some embodiments, the opioid is administered after the surgical procedure.

In some embodiments, the method of treating pain in a patient who will be subjected to a surgical procedure, comprising administering meloxicam to the patient prior to start of the surgical procedure, further comprises administering meloxicam to the patient about every 18 hours to about every 26 hours after administering meloxicam prior to the start of the surgical procedure, until the patient is no longer in need thereof. In some embodiments, the method comprises administering meloxicam to the patient about 18 hours, about 24 hours, about 36 hours, about 48 hours, about 54 hours, about 72 hours, about 90 hours, and/or about 96 hours after administering meloxicam prior to start of the surgical procedure. In particular embodiments, the method comprises administering meloxicam to the patient about every 24 hours after administering meloxicam prior to start of the surgical procedure. In some embodiments, the pain is a moderate to severe pain.

The present disclosure also provides a method of treating acute pain in a patient, said patient being a patient who will be subjected to soft tissue surgery, comprising administering NanoCrystal Colloidal Dispersion (NCD) meloxicam intravenously, over a course of about 15 seconds, to the patient in an amount of about 30 mg at about 30 minutes prior to start of soft tissue surgery. In some embodiments, the method further comprises administering NCD meloxicam to the patient about every 18 hours to about every 26 hours after initially administering NCD meloxicam prior to start of the surgery, until the patient is no longer in need thereof. In some embodiments, the method further comprises administering NCD meloxicam to the patient about 18 hours, about 24 hours, about 36 hours, about 48 hours, about 54 hours, about 72 hours, about 90 hours, and/or about 96 hours after administering NCD meloxicam prior to start of the surgery. In some embodiments, the pain is a moderate to severe pain.

The present disclosure further provides a method of treating acute pain in a patient, said patient being a patient who will be subjected to hard tissue surgery, comprising administering NCD meloxicam intravenously, over a course of about 15 seconds, to the patient in an amount of about 30 mg after the administration of anesthesia and prior to start of surgery. In some embodiments, the method further comprises administering NCD meloxicam to the patient about every 18 hours to about every 26 hours after initially administering NCD meloxicam prior to start of the surgery, until the patient is no longer in need thereof. In some embodiments, the method further comprises administering NCD meloxicam to the about 18 hours, about 24 hours, about 36 hours, about 48 hours, about 54 hours, about 72 hours, about 90 hours, and/or about 96 hours after administering NCD meloxicam prior to start of the surgery. In some embodiments, the pain is a moderate to severe pain.

The present disclosure further provides a method of treating pain in a patient in need thereof, comprising administering meloxicam intravenously to the patient in combination with acetaminophen and/or gabapentin. In some embodiments, the pain is an acute pain. In some embodiments, the pain is a moderate to severe pain. In some embodiments, the meloxicam is present as nanocrystalline meloxicam. In some embodiments, the nanocrystalline meloxicam is in a colloidal dispersion. In some embodiments, meloxicam is administered to the patient in an amount ranging from about 5 mg to about 180 mg or about 15 mg to about 60 mg, such as, for example 30 mg. In some embodiments, meloxicam is administered to the patient intravenously. In some embodiments meloxicam is administered to the patient intravenously over a course of about 5 seconds to about 60 seconds, about 10 second to about 60 seconds, about 15 seconds to about 30 seconds, such as, for example 15 seconds.

In some embodiments, the method comprises administering meloxicam to the patient in combination with acetaminophen. In some embodiments, acetaminophen is administered orally, intravenously or a combination thereof. In some embodiments, acetaminophen is administered in an amount of about 5 mg to about 1 g or about 200 mg to about 800 mg, such as, for example, 650 mg. In some embodiments, the method comprises administering meloxicam to the patient in combination with gabapentin. In some embodiments, gabapentin is administered orally. In some embodiments, gabapentin is administered in an amount of about 200 mg to about 700 mg, such as, for example, about 300 mg or about 600 mg. In some embodiments, meloxicam and acetaminophen and/or gabapentin are administered to the patient concurrently. In other embodiments, meloxicam is administered to the patient within about 2 hours, within about 1 hour or within about 30 minutes of acetaminophen and/or gabapentin administration.

In some embodiments, the patient is a patient who will be subjected to a surgical procedure and wherein meloxicam and acetaminophen and/or gabapentin are administered to the patient prior to start of the surgical procedure. In some embodiments, acetaminophen and/or gabapentin are administered from about 30 to about 90 minutes prior to start of the surgical procedure. In some embodiments, acetaminophen and/or gabapentin are administered before administration of anesthesia. In some embodiments, meloxicam is administered after administration of anesthesia. In some embodiments, the method further comprises administering acetaminophen to the patient after completion of the surgical procedure.

In some embodiments, the surgical procedure is performed on soft tissue, hard tissue or a combination thereof. In some embodiments, the surgical procedure comprises an open surgical procedure. In other embodiments, the surgical procedure comprises a laparoscopic procedure. In some embodiment, the surgical procedure comprises colorectal surgery. In other embodiments, the surgical procedure comprises orthopedic surgery.

In some embodiments, the method further comprises administering an antibiotic, an anti-nausea medication, a medication to treat or prevent excess bleeding, or a combination thereof, to the patient. In some embodiments, the antibiotic is a prophylactic antibiotic. In some embodiments, the anti-nausea medication is ondansetron, dexamethasone, promethazine, scopolamine, or a combination thereof. In some embodiments, the medication to treat or prevent excess bleeding, wherein the medication to treat or prevent excess bleeding is tranexamic acid.

The present disclosure also provides a method treating acute pain in a patient who will be subjected to hard tissue surgery, comprising administering NanoCrystal Colloidal Dispersion (NCD) meloxicam intravenously, over a course about 15 seconds, to the patient in an amount of about 30 mg, in combination with about 650 mg of acetaminophen and about 600 mg of gabapentin, in which acetaminophen and gabapentin are administered at a time within a range of about 30-90 minutes prior to start of surgery, and NCD meloxicam is administered about 30 minutes prior to the start of surgery and after administration of anesthesia. In some embodiments, the method further comprises administering NCD meloxicam to the patient about every 18 hours to about every 26 hours after administering NCD meloxicam prior to the start of the surgery, until the patient is no longer in need thereof. In some embodiments, the method further comprises administering NCD meloxicam to the patient about 18 hours, about 24 hours, about 36 hours, about 48 hours, about 54 hours, about 72 hours, about 90 hours, and/or about 96 hours after administering NCD meloxicam prior to the start of the surgery.

The present disclosure also provides a method of treating acute pain in a patient who will be subjected soft tissue surgery, comprising administering NanoCrystal Colloidal Dispersion (NCD) meloxicam intravenously, over a course of 15 seconds, to the patient in an amount of about 30 mg, in combination with about 650 mg of acetaminophen about 300 mg of gabapentin, in which acetaminophen and gabapentin are administered at a time within a range of about 30-90 minutes prior to start of surgery, and NCD meloxicam is administered about 30 minutes prior to start of surgery. In some embodiments, the method further comprises administering NCD meloxicam to the patient about every 18 hours to about every 26 hours after administering NCD meloxicam prior to the start of the surgery, until the patient is no longer in need thereof. In some embodiments, the method further comprises administering NCD meloxicam to the patient about 18 hours, about 24 hours, about 36 hours, about 48 hours, about 54 hours, about 72 hours, about 90 hours, and/or about 96 after administering NCD meloxicam prior to the start of the surgery.

In some embodiments, the method of treating pain in a patient comprising administering intravenous meloxicam, acetaminophen and gabapentin in combination to a patient prior to start of a surgical procedure further comprises administering acetaminophen every 6 hours subsequent to administration of acetaminophen prior to start of the surgical procedure. In some embodiments, meloxicam is administered about every 18 hours to about every 26 hours subsequent to administration of meloxicam prior to start of the surgical procedure. In some embodiments, the pain is a moderate to severe pain.

In some embodiments, the methods of the present disclosure further comprise administering an analgesic concurrently with meloxicam. In some embodiments, the analgesic is administered concurrently with meloxicam prior to surgery. In some embodiments, the analgesic is administered concurrently with meloxicam subsequent to surgery. In some embodiments, the analgesic is an opioid. In some embodiments, the opioid is administered subsequent to surgery.

Other objects, features and advantages of the present disclosure will become apparent from the following detailed description. It should be understood, however, that the detailed description and the specific examples, while indicating preferred embodiments, are given by way of illustration only, since various changes and modifications within the spirit and scope of the disclosure will become apparent to those skilled in the art from this detailed description.

### BRIEF DESCRIPTION OF THE DRAWINGS

FIG. 1 shows summary of Opioid Consumption at 6 Hour Intervals Following Surgery in the mITT population. The efficacy population, referred to as the modified intent-to-treat (mITT) population, included all subjects who received at least one injection of study drug and underwent their scheduled surgery. The efficacy/mITT population was used for all efficacy assessments.

FIG. 2 shows the Kaplan-Meier Survival Curve for Time to First Opioid Rescue in the mITT population.

### DETAILED DESCRIPTION

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like and reference to “the method” includes reference to equivalent steps and/or methods known to those skilled in the art, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application. Generally the term “about”, as used herein in references to a measurable value such as an amount of weight, time, dose, etc. is meant to encompass values within an acceptable degree of variability in the art. In some embodiments, degree of variability is based on FDA guidelines.

As used herein, “meloxicam” refers to 4-hydroxy-2-methyl-N-(5-methyl-2-thiazolyl)-2H-1,2-benzothiazine-3-carboxamide-1,1-dioxide, which has the structure as depicted below. The molecular weight is 351.4. Its molecular formula is C₁₄H₁₃N₃O₄S₂.

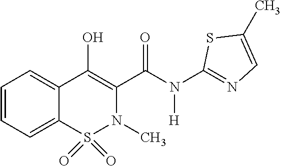 [SMILES: Cc1cnc(NC(=O)C2=C(O)c3ccccc3S(=O)(=O)N2C)s1]

As used herein, the term “bolus dose” refers to a discrete amount of a medication or a drug, e.g., meloxicam, which is given within a specific time. The specific time over which the bolus dose is administered (also referred to herein as the infusion rate) may be any suitable time which provides rapid onset of action (i.e., pain relief) and which does not cause significant injection site pain, such as a significant burning sensation. In some embodiments, the infusion time may be about 1 minute or less, e.g., about 30 seconds or about 15 seconds.

As used herein, “treatment” is an approach for obtaining beneficial or desired clinical results. For purposes of this invention, beneficial or desired clinical results include, but are not limited to, one or more of the following: improvement in any aspect of the pain including lessening severity, alleviation of one or more symptoms associated with pain including any aspect of pain (such as resting pain and/or mechanically-induced pain, shortening duration of pain, and/or reduction of pain sensitivity or sensation), reducing the incidence of, managing, ameliorating, preventing, and/or the delaying the development or progression of pain.

The term “effective amount” or “therapeutically effective amount” refers to the amount of an agent that is sufficient to achieve an outcome, for example, to effect beneficial or desired results. The therapeutically effective amount may vary depending upon one or more of: the subject and disease condition being treated, the weight and age of the subject, the severity of the disease condition, the manner of administration and the like.

The term “concurrent” or “concurrently” refers to administering two or more drugs close in time to each other. In some embodiments, the two or more concurrently administered drugs are administered within 1 hour, within about 30 min, within about 15 minutes, within about 10 minutes, within about 5 minutes of each other. In some embodiments, the two or more concurrently administered drugs are administered simultaneously.

### Therapeutic Use

While oral administration of meloxicam is approved for treating inflammation (e.g., osteoarthritis and rheumatoid arthritis), currently available oral formulations of meloxicam are known to have a slow onset of action due to poor solubility of meloxicam. The slow onset of action of oral meloxicam has rendered meloxicam not appropriate for acute pain management (e.g., mild to moderate pain and/or moderate to severe pain).

The inventors discovered that an intravenous formulation of meloxicam may be administered prior to a surgical procedure and/or in combination with additional therapeutic agents to provide a rapid onset of action of meloxicam that is critical for treatment of acute pain, such as surgical pain. Meloxicam nanocrystals significantly improves the solubility of the meloxicam, allowing for higher concentrations of meloxicam to be administered intravenously compared to an otherwise similar formulation in which meloxicam is not prepared as nanocrystals. Specifically, the inventors found that a meloxicam dose of about 5 mg to about 200 mg can provide a rapid onset of action of meloxicam while being efficacious and safe for the treatment of acute pain (e.g., mild to moderate pain and/or moderate to severe pain). In contrast to other intravenous NSAIDs such as ibuprofen and ketorolac, meloxicam nanocrystals can be safely administered intravenously without causing injection site pain. In addition, the inventors found that a bolus dose given over about 60 seconds (e.g., about 1 to about 60 seconds, about 1 to about 30 seconds, about 15 to about 30 seconds, etc.) was safe and effective for the treatment of pain. In some embodiments, the administration of intravenous meloxicam provided pain relief to the patient within about 15 minutes to within about 24 hours, for example, within about 15 minutes, within about 30 minutes, within about 1 hour, within about 2 hours, within about 3 hours, within about 4 hours, within about 5 hours, within about 6 hours, within about 12 hours, within about 18 hours, within about 24 hours, inclusive of all the values and subranges therebetween.

In one embodiment, the methods disclosed herein comprise administering to the patient a dose of meloxicam intravenously, wherein the meloxicam is at a dose of about 30 mg. In some embodiments, the methods disclosed herein comprise administering to the patient a dose of meloxicam intravenously, wherein the meloxicam is at a concentration of about 30 mg/mL. In one embodiment, the intravenous dose is a bolus dose. In some embodiments, the drug doses may be adjusted for patients based on the surgery that is to be performed on the patient, age of the patient and the clinical condition of the patient. In some embodiments, the meloxicam is administered intramuscularly.

In one embodiment, the meloxicam is in a form of meloxicam nanocrystals. In another embodiment, meloxicam nanocrystals are formed using Alkermes NanoCrystal™ technology. See U.S. Pat. No. 8,512,727 which is hereby incorporated by reference in its entirety for all purposes.

In one embodiment of the method as disclosed herein, the IV dose (including a bolus dose) of meloxicam is administered to the patient over the course of about 1 to about 60 seconds, including all values and subranges therebetween. That is, the IV dose of meloxicam may be administered to a patient in about 1 second, about 2 seconds, about 3 seconds, about 4 seconds, about 5 seconds, about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 21 seconds, about 22 seconds, about 23 seconds, about 24 seconds, about 25 seconds, about 26 seconds, about 27 seconds, about 28 seconds, about 29 seconds, about 30 seconds, about 31 seconds, about 32 seconds, about 33 seconds, about 34 seconds, about 35 seconds, about 36 seconds, about 37 seconds, about 38 seconds, about 39 seconds, about 40 seconds, about 41 seconds, about 42 seconds, about 43 seconds, about 44 seconds, about 45 seconds, about 46 seconds, about 47 seconds, about 48 seconds, about 49 seconds, about 50 seconds, about 51 seconds, about 52 seconds, about 53 seconds, about 54 seconds, about 55 seconds, about 56 seconds, about 57 seconds, about 58 seconds, about 59 seconds, or about 60 seconds, or any ranges between these values.

For example, in some embodiments, the IV dose (including a bolus dose) of meloxicam is administered to the patient over the course of about 5 to about 45 seconds. In other embodiments, the IV dose of meloxicam is administered to the patient over the course of about 10 to about 40 seconds. In still other embodiments, the IV dose of meloxicam is administered to the patient over the course of about 15 to about 35 seconds. In some embodiments, the IV dose of meloxicam is administered to the patient over the course of about 10 to about 30 seconds. In certain embodiments, the IV dose of meloxicam is administered to the patient over the course of about 15 to about 30 seconds. In one embodiment, the IV dose of meloxicam is administered to the patient over about 15 seconds.

The infusion rates of the present disclosure are significantly quicker than the FDA-approved infusion time of CALDOLOR® (an intravenous formulation of the NSAID ibuprofen), which requires at least 30 minutes. See CALDOLOR® Prescribing Information. Similarly, the infusion rates of the present disclosure are also significantly faster than infusion rates for OFIRMEV® (an intravenous formulation of acetaminophen), which requires a 15 minute infusion rate. See OFIRMEV® Prescribing Information. Whereas intravenous formulations of ibuprofen and acetaminophen cause injection site pain when administered at a rate that is faster than 15 minutes and 30 minutes, respectively, the present formulations were surprisingly discovered not to cause such injection site pain when administered in a IV dose (including a bolus dose).

Further, the inventors discovered that an injection of meloxicam within seconds, according to the methods disclosed herein, achieves fast onset of analgesics which is critical for management of acute pain, such as post-surgical pain. For example, in one embodiment, the dose of meloxicam administered intravenously to a patient can provide pain relief within about 10 minutes. This rapid onset of pain relief provided by the methods of the present disclosure is a substantial improvement from available intravenous NSAIDs, such as ketorolac which can take up to 30 minutes for the onset of pain relief. See Ketorolac Tromethamine Injection Prescribing Information. In other embodiments, the dose of meloxicam can be administered intravenously to a patient prior to surgery and advantageously treat post-surgical pain. In further or alternative embodiments, meloxicam can be administered in combination with other therapeutic agents to provide pain relief.

Moreover, unlike the previously reported NSAID injections which resulted in high injection site pain adverse effects (e.g., 16%-24% reported), the inventors found that the injection methods for administration of meloxicam disclosed herein is safe and efficacious, as only 2% of patients receiving a dose of intravenous meloxicam reported injection site pain.

In one embodiment of the methods disclosed herein, the dose of meloxicam is in the range of from about 1 mg to about 250 mg, inclusive of all values and subranges therebetween. That is, the dose of meloxicam may be about 1 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 205 mg, about 210 mg, about 215 mg, about 220 mg, 225 mg, about 230 mg, 235 mg, about 240 mg, 245 mg, or about 250 mg, or any ranges between these values.

In one embodiment, the dose of meloxicam is in the range of from about 5 mg to about 200 mg. In some embodiments, the dose of meloxicam is in the range of from about 15 mg to about 180 mg. In some embodiments, the dose of meloxicam is in the range of from about 15 mg to about 100 mg. In other embodiments, the dose of meloxicam is in the range of from about 15 mg to about 80 mg. In some embodiments, the dose of meloxicam is in the range of from about 20 mg to about 70 mg. In some embodiments, the dose of meloxicam is in the range of from about 30 mg to about 60 mg. In some embodiments, the dose of meloxicam is about 30 mg. In another embodiment, the dose of meloxicam is about 60 mg.

In some embodiments, the intravenous meloxicam is formulated at a concentration of from about 10 mg/mL to about 50 mg/mL, e.g., about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, and about 60 mg/mL, inclusive of all values and subranges therebetween. In particular embodiments, the intravenous meloxicam is formulated at a concentration of about 30 mg/mL.

In some embodiments, the dose of meloxicam as disclosed herein is administered once a day, twice a day, three times a day, every other day, or at a frequency deemed appropriate by a physician. In one embodiment, the dose of meloxicam is administered once a day intravenously. In some embodiments, meloxicam is administered every 18-26 hours until the patient is no longer in need thereof. As used herein, a “patient is no longer in need thereof” when the pain has subsided or the patient is discharged from the hospital. In some embodiments, meloxicam is administered intravenously once every 12 hours, once every 18 hours, once every 24 hours, once every 36 hours, once every 48 hours or at a frequency deemed appropriate by a physician. In particular embodiments, meloxicam is administered once every 24 hours.

In some embodiments, the dose of meloxicam as disclosed herein can be administered once a day for 1 day, 2 days, 3 days, 4 days, 5 days, 6 days, 7 days, 8 days, 9 days, 10 days, 11 days, 12 days, 13 days, 14 days, or at a duration and frequency deemed appropriate by a physician.

In one embodiment, a single dose, including a bolus dose, as disclosed herein can provide a rapid treatment which lasts for about 12 hours to about 48 hours. In one embodiment, a single dose as disclosed herein can provide a rapid treatment which lasts for about 24 hours. The ability for the presently disclosed meloxicam formulation to provide treatment lasting about 24 hours is a significant improvement over previously approved NSAID IV treatments, such as CALDOLOR® which requires infusion every 6 hours. See CALDOLOR® Prescribing Information.

In any of the methods disclosed herein, meloxicam can be administered for treatment of pain or for pain management. In one embodiment, meloxicam can be administered for the treatment or management of moderate to severe pain. In one embodiment, meloxicam can be administered for the treatment or management of mild to moderate pain. In one embodiment, the pain management is for a human patient. In one embodiment, the human patient is an adult.

### Formulations

In one embodiment, the dose for an IV injection or an IV infusion disclosed herein can comprise one or more pharmaceutically acceptable excipients or carriers known to one skilled in the art.

In one embodiment, a pharmaceutically acceptable excipient for the dose for an IV injection or an IV infusion can include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, sodium deoxycholate (deoxycholic acid), starch tragacanth, sucrose or xanthan gum.

In one embodiment, the dose of meloxicam disclosed herein for injection or infusion can be formulated in liquid carriers such as, water, dextrose in water, glucose in water, vegetable oil, alcohol, polyethylene glycol, propylene glycol, or glycerin. In one embodiment, the dose of meloxicam disclosed herein for injection is formulated in sterile water.

In one embodiment, the dose of meloxicam is in a form of aqueous dispersion.

In one embodiment of the method as disclosed herein, the dose of meloxicam is present in a volume of from about 0.5 mL to about 4 mL, inclusive of all values and subranges therebetween. That is, the IV dose (including a bolus dose) of meloxicam is present in a volume of about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, about 1.5 mL, about 1.6 mL, about 1.7 mL, about 1.8 mL, about 1.9 mL, about 2.0 mL, about 2.1 mL, about 2.2 mL, about 2.3 mL, about 2.4 mL, about 2.5 mL, about 2.6 mL, about 2.7 mL, about 2.8 mL, about 2.9 mL, about 3.0 mL, about 3.1 mL, about 3.2 mL, about 3.3 mL, about 3.4 mL, about 3.5 mL, about 3.6 mL, about 3.7 mL, about 3.8 mL, about 3.9 mL, or about 4.0 mL, or any ranges between these values. In another embodiment, the dose of meloxicam is present in a volume of about 1 mL.

In one embodiment of the method as disclosed herein, the dose of meloxicam is present at a concentration of about 30 mg/mL. That is, the dose of meloxicam can be present at a concentration between about 28.5 mg/mL and about 31.5 mg/mL or any subranges between the two values. In one embodiment, the dose of meloxicam can be present at a concentration of about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, or about 38 mg/mL, inclusive of all values and subranges therebetween. In one embodiment, the dose of meloxicam as disclosed herein can be a bolus dose.

In one embodiment, the dose of meloxicam is present at a concentration of about 30 mg/mL as a single use vial.

As previously noted, meloxicam has poor water solubility, which is one of the main reasons oral administration is not suitable for treatment of acute pain. Further, due to meloxicam's poor water solubility, it is challenging to provide an injectable formulation that is sufficiently concentrated so that the formulation can be injected to patients in seconds in order to achieve rapid onset of pain relief without causing injection site pain. However, the inventors were able to increase the meloxicam concentration to 30 mg/mL by using meloxicam nanocrystals. This is a 20% increase in the concentration as compared to an otherwise similar formulation in which meloxicam is not prepared as nanocrystals, which is substantial considering meloxicam is poorly water soluble. The concentration of meloxicam as disclosed herein is critical to providing an IV dose and achieving rapid onset of pain relief without causing injection site pain. At concentrations of meloxicam which are higher than those disclosed herein, the drugs may crystalize out of solution, which may interfere with the injectability and/or syringeability of the formulation. At concentrations of meloxicam which are lower than those disclosed herein, the larger volumes of carrier preclude administration within the time ranges specified herein, and thereby cannot achieve rapid onset of pain relief.

In one embodiment, the dose of meloxicam as disclosed herein is used with dilution. In one embodiment, the dose of meloxicam as disclosed herein is used without dilution. In one embodiment, the 30 mg/mL dose of meloxicam is used without dilution. In one embodiment, the 30 mg/mL dose of meloxicam is not added to an IV solution or an IV fluid bag. That is, the 30 mg/mL dose of meloxicam as disclosed herein is administered to a patient in need thereof as 30 mg/mL.

### Pharmacokinetics

In one embodiment, a single 30 mg/mL bolus dose provides an average blood plasma Cₘₐₓ within about 80% to about 125% of the range of from about 4000 ng/mL to about 11000 ng/mL in a patient after intravenous administration of 30 mg of meloxicam, inclusive of all values and subranges therebetween. That is, a single 30 mg/mL bolus dose can provide plasma Cₘₐₓ of about 3000 ng/mL, about 3100 ng/mL, about 3200 ng/mL, about 3300 ng/mL, about 3400 ng/mL, about 3500 ng/mL, about 3600 ng/mL, about 3700 ng/mL, about 3800 ng/mL, about 3900 ng/mL, about 4000 ng/mL, about 4100 ng/mL, about 4200 ng/mL, about 4300 ng/mL, about 4400 ng/mL, about 4500 ng/mL, about 4600 ng/mL, about 4700 ng/mL, about 4800 ng/mL, about 4900 ng/mL, about 5000 ng/mL, about 5100 ng/mL, about 5200 ng/mL, about 5300 ng/mL, about 5400 ng/mL, about 5500 ng/mL, about 5600 ng/mL, about 5700 ng/mL, about 5800 ng/mL, about 5900 ng/mL, about 6000 ng/mL, about 6100 ng/mL, about 6200 ng/mL, about 6300 ng/mL, about 6400 ng/mL, about 6500 ng/mL, about 6600 ng/mL, about 6700 ng/mL, about 6800 ng/mL, about 6900 ng/mL, about 7000 ng/mL, about 7100 ng/mL, about 7200 ng/mL, about 7300 ng/mL, about 7400 ng/mL, about 7500 ng/mL, about 7600 ng/mL, about 7700 ng/mL, about 7800 ng/mL, about 7900 ng/mL, about 8000 ng/mL, about 8100 ng/mL, about 8200 ng/mL, about 8300 ng/mL, about 8400 ng/mL, about 8500 ng/mL, about 8600 ng/mL, about 8700 ng/mL, about 8800 ng/mL, about 8900 ng/mL, about 9000 ng/mL, about 9100 ng/mL, about 9200 ng/mL, about 9300 ng/mL, about 9400 ng/mL, about 9500 ng/mL, about 9600 ng/mL, about 9700 ng/mL, about 9800 ng/mL, about 9900 ng/mL, about 10000 ng/mL, about 10100 ng/mL, about 10200 ng/mL, about 10300 ng/mL, about 10400 ng/mL, about 10500 ng/mL, about 10600 ng/mL, about 10700 ng/mL, about 10800 ng/mL, about 10900 ng/mL, about 11000 ng/mL, about 11100 ng/mL, about 11200 ng/mL, about 11300 ng/mL, about 11400 ng/mL, about 11500 ng/mL, about 11600 ng/mL, about 11700 ng/mL, about 11800 ng/mL, about 11900 ng/mL, about 12000 ng/mL, about 12100 ng/mL, about 12200 ng/mL, about 12300 ng/mL, about 12400 ng/mL, about 12500 ng/mL, about 12600 ng/mL, about 12700 ng/mL, about 12800 ng/mL, about 12900 ng/mL, about 13000 ng/mL, about 13100 ng/mL, about 13200 ng/mL, about 13300 ng/mL, about 13400 ng/mL, and about 13500 ng/mL, or any values or ranges between above values, in a patient.

In one embodiment, a 1 mL bolus of 30 mg/mL provides an average plasma Cₘₐₓ within about 80% to about 125% of the range of from about 5642.9±1009.0 ng/mL in a patient after intravenous administration of intravenous meloxicam, inclusive of all values and subranges therebetween. In one embodiment, a single 30 mg/mL bolus dose provides an average plasma Cₘₐₓ within the range of from about 3707.1 ng/mL to about 8314.9 ng/mL in a patient after intravenous administration of intravenous meloxicam, inclusive of all values and subranges therebetween. In one embodiment, a single 30 mg/mL bolus dose provides an average plasma Cₘₐₓ within about 80% to about 125% of the range of from about 4000 ng/mL to about 7000 ng/mL in a patient after intravenous administration of 30 mg of meloxicam, inclusive of all values and subranges therebetween. In one embodiment, a single 30 mg/mL bolus dose provides an average plasma Cₘₐₓ within about 80% to about 125% of the range of from about 4600 ng/mL to about 6700 ng/mL in a patient after intravenous administration of intravenous meloxicam, inclusive of all values and subranges therebetween. In one embodiment, a single 30 mg/mL bolus dose provides an average plasma Cₘₐₓ within about 80% to about 125% of the range of from about 5000 ng/mL to about 6000 ng/mL in a patient after intravenous administration of 30 mg of meloxicam, inclusive of all values and subranges therebetween.

In one embodiment, a single 30 mg/mL bolus dose provides plasma Cₘₐₓ within about 80% to about 125% of the range of from about 7972.5±2579.0 ng/mL in a patient after intravenous administration of 30 mg of meloxicam, inclusive of all values and subranges therebetween. In one embodiment, a single 30 mg/mL bolus dose provides plasma Cₘₐₓ within the range of from about 4,312.1 ng/mL to about 13,190.5 ng/mL in a patient after intravenous administration of 30 mg of meloxicam, inclusive of all values and subranges therebetween. In one embodiment, a single 30 mg/mL bolus dose provides an average plasma Cₘₐₓ within about 80% to about 125% of the range of from about 5000 ng/mL to about 11000 ng/mL in a patient after intravenous administration of 30 mg of meloxicam, inclusive of all values and subranges therebetween. In one embodiment, a single 30 mg/mL bolus dose provides an average plasma Cₘₐₓ within about 80% to about 125% of the range of from about 5500 ng/mL to about 10500 ng/mL in a patient after intravenous administration of 30 mg of meloxicam, inclusive of all values and subranges therebetween. In one embodiment, a single 30 mg/mL bolus dose provides an average plasma Cₘₐₓ within about 80% to about 125% of the range of from about 7000 ng/mL to about 9000 ng/mL in a patient after intravenous administration of intravenous meloxicam, inclusive of all value sand subranges therebetween.

In one embodiment, a repeat dose (e.g., administered once daily) of a 30 mg/mL bolus dose provides plasma Cₘₐₓ (e.g., a steady state Cₘₐₓ) within about 80% to about 125% of the range of from about 10632.5±4729.8 ng/mL in a patient after intravenous administration of 30 mg of meloxicam, inclusive of all values and subranges therebetween. In one embodiment, a repeat dose of a 30 mg/mL bolus dose provides plasma Cₘₐₓ within the range of from about 4,722.2 ng/mL to about 19,202.9 ng/mL in a patient after intravenous administration of 30 mg of meloxicam, inclusive of all values and subranges therebetween. That is, a repeat dose of 30 mg/mL bolus dose can provide plasma Cₘₐₓ of about 4500 ng/mL, about 4600 ng/mL, about 4700 ng/mL, about 4800 ng/mL, about 4900 ng/mL, about 5000 ng/mL, about 5100 ng/mL, about 5200 ng/mL, about 5300 ng/mL, about 5400 ng/mL, about 5500 ng/mL, about 5600 ng/mL, about 5700 ng/mL, about 5800 ng/mL, about 5900 ng/mL, about 6000 ng/mL, about 6100 ng/mL, about 6200 ng/mL, about 6300 ng/mL, about 6400 ng/mL, about 6500 ng/mL, about 6600 ng/mL, about 6700 ng/mL, about 6800 ng/mL, about 6900 ng/mL, about 7000 ng/mL, about 7100 ng/mL, about 7200 ng/mL, about 7300 ng/mL, about 7400 ng/mL, about 7500 ng/mL, about 7600 ng/mL, about 7700 ng/mL, about 7800 ng/mL, about 7900 ng/mL, about 8000 ng/mL, about 8100 ng/mL, about 8200 ng/mL, about 8300 ng/mL, about 8400 ng/mL, about 8500 ng/mL, about 8600 ng/mL, about 8700 ng/mL, about 8800 ng/mL, about 8900 ng/mL, about 9000 ng/mL, about 9100 ng/mL, about 9200 ng/mL, about 9300 ng/mL, about 9400 ng/mL, about 9500 ng/mL, about 9600 ng/mL, about 9700 ng/mL, about 9800 ng/mL, about 9900 ng/mL, about 10000 ng/mL, about 10100 ng/mL, about 10200 ng/mL, about 10300 ng/mL, about 10400 ng/mL, about 10500 ng/mL, about 10600 ng/mL, about 10700 ng/mL, about 10800 ng/mL, about 10900 ng/mL, about 11000 ng/mL, about 11100 ng/mL, about 11200 ng/mL, about 11300 ng/mL, about 11400 ng/mL, about 11500 ng/mL, about 11600 ng/mL, about 11700 ng/mL, about 11800 ng/mL, about 11900 ng/mL, about 12000 ng/mL, about 12100 ng/mL, about 12200 ng/mL, about 12300 ng/mL, about 12400 ng/mL, about 12500 ng/mL, about 12600 ng/mL, about 12700 ng/mL, about 12800 ng/mL, about 12900 ng/mL, about 13000 ng/mL, about 13100 ng/mL, about 13200 ng/mL, about 13300 ng/mL, about 13400 ng/mL, about 13500 ng/mL, about 13600 ng/mL, about 13700 ng/mL, about 13800 ng/mL, about 13900 ng/mL, about 14000 ng/mL, about 14100 ng/mL, about 14200 ng/mL, about 14300 ng/mL, about 14400 ng/mL, about 14500 ng/mL, about 14600 ng/mL, about 14700 ng/mL, about 14800 ng/mL, about 14900 ng/mL, about 15000 ng/mL, about 15100 ng/mL, about 15200 ng/mL, about 15300 ng/mL, about 15400 ng/mL, about 15500 ng/mL, about 15600 ng/mL, about 15700 ng/mL, about 15800 ng/mL, about 15900 ng/mL, about 16000 ng/mL, about 16100 ng/mL, about 16200 ng/mL, about 16300 ng/mL, about 16400 ng/mL, about 16500 ng/mL, about 16600 ng/mL, about 16700 ng/mL, about 16800 ng/mL, about 16900 ng/mL, about 17000 ng/mL, about 17100 ng/mL, about 17200 ng/mL, about 17300 ng/mL, about 17400 ng/mL, about 17500 ng/mL, about 17600 ng/mL, about 17700 ng/mL, about 17800 ng/mL, about 17900 ng/mL, about 18000 ng/mL, about 18100 ng/mL, about 18200 ng/mL, about 18300 ng/mL, about 18400 ng/mL, about 18500 ng/mL, about 18600 ng/mL, about 18700 ng/mL, about 18800 ng/mL, about 18900 ng/mL, about 19000 ng/mL, about 19100 ng/mL, about 19200 ng/mL, about 19300 ng/mL, about 19400 ng/mL, about 19500 ng/mL, about 19600 ng/mL, about 19700 ng/mL, about 19800 ng/mL, about 19900 ng/mL, or about 12000 ng/mL, or any values or ranges between above values, in a patient.

In one embodiment, a repeat dose of a 30 mg/mL bolus dose provides plasma Cₘₐₓ within about 80% to about 125% of the range of from about 5000 ng/mL to about 20000 ng/mL in a patient after intravenous administration of 30 mg of meloxicam, inclusive of all values and subranges therebetween. In one embodiment, a repeat dose of 30 mg/mL bolus dose provides an average plasma Cₘₐₓ within about 80% to about 125% of the range of from about 7000 ng/mL to about 18000 ng/mL in a patient after intravenous administration of 30 mg of meloxicam, inclusive of all values and subranges therebetween. In one embodiment, a repeat dose of 30 mg/mL bolus dose provides an average plasma Cₘₐₓ within m about 80% to about 125% of the range of from about 8000 ng/mL to about 13000 ng/mL in a patient after intravenous administration of 30 mg of meloxicam, inclusive of all values and subranges therebetween.

In one embodiment, a single 30 mg/mL bolus dose provides an average plasma AUC_{inf} within about 80% to about 125% of the range of from about 55,000 ng*hr/mL to about 190,000 ng*hr/mL in a patient after intravenous administration of 30 mg of meloxicam, inclusive of all values and subranges therebetween. That is, a single 30 mg/mL bolus dose can provide an average plasma AUC_{inf} of about 40,000 ng*hr/mL, about 45,000 ng*hr/mL, about 50,000 ng*hr/mL, about 55,000 ng*hr/mL, about 60,000 ng*hr/mL, about 65,000 ng*hr/mL, about 70,000 ng*hr/mL, about 75,000 ng*hr/mL, about 80,000 ng*hr/mL, about 85,000 ng*hr/mL, about 90,000 ng*hr/mL, about 95,000 ng*hr/mL, about 100,000 ng*hr/mL, about 105,000 ng*hr/mL, about 110,000 ng*hr/mL, about 115,000 ng*hr/mL, about 120,000 ng*hr/mL, about 125,000 ng*hr/mL, about 130,000 ng*hr/mL, about 135,000 ng*hr/mL, about 140,000 ng*hr/mL, about 145,000 ng*hr/mL, about 150,000 ng*hr/mL, about 155,000 ng*hr/mL, about 160,000 ng*hr/mL, about 165,000 ng*hr/mL, about 170,000 ng*hr/mL, about 175,000 ng*hr/mL, about 180,000 ng*hr/mL, about 185,000 ng*hr/mL, about 190,000 ng*hr/mL, about 195,000 ng*hr/mL, about 200,000 ng*hr/mL, about 205,000 ng*hr/mL, about 210,000 ng*hr/mL, about 215,000 ng*hr/mL, about 220,000 ng*hr/mL, about 225,000 ng*hr/mL, about 230,000 ng*hr/mL, about 235,000 ng*hr/mL, and about 240,000 ng*hr/mL, or any values or ranges between above values, in a patient.

In one embodiment, a single 30 mg/mL bolus dose provides an average plasma AUC_{inf} of within about 80% to about 125% of the range of from about 107508.7±34443.0 ng*hr/mL in a patient after intravenous administration of 30 mg of meloxicam, inclusive of all values and subranges therebetween. In one embodiment, a single 30 mg/mL bolus dose provides an average plasma AUC_{inf} within the range of from about 58,452.6 ng*hr/mL to about 177,440.0 ng*hr/mL in a patient after intravenous administration of 30 mg of meloxicam, inclusive of all values and subranges therebetween. In one embodiment, a single 30 mg/mL bolus dose provides an average plasma AUC_{inf} within about 80% to about 125% of the range of from about 121437.6±64505.6 ng*hr/mL in a patient after intravenous administration of 30 mg of meloxicam, inclusive of all values and subranges therebetween. In one embodiment, a single 30 mg/mL bolus dose provides an average plasma AUC_{inf} within the range of from about 45,545.6 ng*hr/mL to about 232,429.0 ng*hr/mL in a patient after intravenous administration of 30 mg of meloxicam, inclusive of all values and subranges therebetween. In one embodiment, a single 30 mg/mL bolus dose provides an average plasma AUC_{inf} of about 70,000 ng*hr/mL to about 190,000 ng*hr/mL in a patient after intravenous administration of 30 mg of meloxicam, inclusive of all values and subranges therebetween. In one embodiment, a single 30 mg/mL bolus dose provides an average plasma AUC_{inf} within the range of from about 80% to about 125% of about 70,000 ng*hr/mL to about 140,000 ng*hr/mL in a patient after intravenous administration of 30 mg of meloxicam, inclusive of all values and subranges therebetween. In one embodiment, a single 30 mg/mL bolus dose provides an average plasma AUC_{inf} within the range of from about 80% to about 125% of about 75,000 ng*hr/mL to about 130,000 ng*hr/mL in a patient after intravenous administration of 30 mg of meloxicam, inclusive of all values and subranges therebetween. In one embodiment, a single 30 mg/mL bolus dose provides an average plasma AUC_{inf} within the range of from about 80% to about 125% of about 85,000 ng*hr/mL to about 120,000 ng*hr/mL in a patient after intravenous administration of 30 mg of meloxicam, inclusive of all values and subranges therebetween. In one embodiment, a single 30 mg/mL bolus dose provides an average plasma AUC_{inf} within the range of from about 80% to about 125% of about 55,000 ng*hr/mL to about 190,000 ng*hr/mL in a patient after intravenous administration of 30 mg of meloxicam, inclusive of all values and subranges therebetween. In one embodiment, a single 30 mg/mL bolus dose provides an average plasma AUC_{inf} within about 80% to about 125% of the range of from about 80,000 ng*hr/mL to about 160,000 ng*hr/mL in a patient after intravenous administration of 30 mg of meloxicam, inclusive of all values and subranges therebetween. In one embodiment, a single 30 mg/mL bolus dose provides an average plasma AUC_{inf} of about within about 80% to about 125% the range of from about 100,000 ng*hr/mL to about 140,000 ng*hr/mL in a patient after intravenous administration of 30 mg of meloxicam, inclusive of all values and subranges therebetween.

In one embodiment, a repeat dose (e.g., more than one dose) of a 30 mg/mL bolus dose provides plasma AUC_{inf} within about 80% to about 125% of the range of from about 297,771.6±241,604.01 ng*hr/mL in a patient after intravenous administration of 30 mg of meloxicam, inclusive of all values and subranges therebetween. In one embodiment, a repeat dose of a 30 mg/mL bolus dose provides plasma AUC_{inf} within the range of from about 44,934.1 ng*hr/mL to about 674,219.5 ng*hr/mL in a patient after intravenous administration of 30 mg of meloxicam, inclusive of all values and subranges therebetween. In one embodiment, a repeat dose of a 30 mg/mL bolus dose provides an average plasma AUC_{inf} within about 80% to about 125% of the range of from about 55,000 ng*hr/mL to about 540,000 ng*hr/mL in a patient after intravenous administration of 30 mg of meloxicam, inclusive of all values and subranges therebetween. In one embodiment, a repeat dose of a 30 mg/mL bolus dose provides an average plasma AUC_{inf} within about 80% to about 125% of the range of from about 80,000 ng*hr/mL to about 500,000 ng*hr/mL in a patient after intravenous administration of 30 mg of meloxicam, inclusive of all values and subranges therebetween. In one embodiment, a repeat dose of a 30 mg/mL bolus dose provides an average plasma AUC_{inf} of about within about 80% to about 125% the range of from about 100,000 ng*hr/mL to about 450,000 ng*hr/mL in a patient after intravenous administration of 30 mg of meloxicam, inclusive of all values and subranges therebetween. In one embodiment, a repeat dose of a 30 mg/mL bolus dose provides an average plasma AUC_{inf} of about within about 80% to about 125% the range of from about 150,000 ng*hr/mL to about 400,000 ng*hr/mL in a patient after intravenous administration of 30 mg of meloxicam, inclusive of all values and subranges therebetween. In one embodiment, a repeat dose of a 30 mg/mL bolus dose provides an average plasma AUC_{inf} of about within about 80% to about 125% the range of from about 200,000 ng*hr/mL to about 350,000 ng*hr/mL in a patient after intravenous administration of 30 mg of meloxicam, inclusive of all values and subranges therebetween. In one embodiment, a repeat dose of a 30 mg/mL bolus dose provides an average plasma AUC_{inf} of about within about 80% to about 125% the range of from about 250,000 ng*hr/mL to about 325,000 ng*hr/mL in a patient after intravenous administration of 30 mg of meloxicam, inclusive of all values and subranges therebetween.

In one embodiment, steady state can be achieved upon repeat dose of a 30 mg bolus dose administered intravenously once daily for 7 days.

In one embodiment, a single 30 mg/mL bolus IV dose provides an average plasma Tₘₐₓ of about 0.05 h to about 0.20 h in a patient after intravenous administration of 30 mg of meloxicam, inclusive of all values and subranges therebetween. That is, a single 30 mg/mL bolus IV injection can provide an average plasma Tₘₐₓ of about 0.05 h, about 0.06 h, about 0.07 h, about 0.08 h, about 0.09 h, about 0.10 h, about 0.11 h, about 0.12 h, about 0.13 h, about 0.14 h, about 0.15 h, about 0.16 h, about 0.17 h, about 0.18 h, about 0.19 h, or about 0.20 h, or any values or ranges between above values, in a patient.

In one embodiment, a single 30 mg/mL bolus dose provides an average plasma Tₘₐₓ of about 0.08 h to about 0.16 h in a patient after intravenous administration of 30 mg of meloxicam, inclusive of all values and subranges therebetween. In one embodiment, a single 30 mg/mL bolus dose provides an average plasma Tₘₐₓ of about 0.10 h to about 0.14 h in a patient after intravenous administration of 30 mg of meloxicam, inclusive of all values and subranges therebetween.

An orally administered meloxicam has a prolonged absorption, with a mean plasma Tₘₐₓ of about 5-7 hours following administration. The methods as disclosed herein provides significantly faster Tₘₐₓ, e.g., about 0.08 h to about 0.16 h following administration, which is indicative of rapid onset and fast absorption.

In one embodiment, the method as disclosed herein can provide meloxicam peak analgesic effect within about 30 minutes to about 60 minutes. That is, the administration of 30 mg/mL bolus IV injection of meloxicam can provide peak analgesic effect in about 30 minutes, about 35 minutes, about 40 minutes, about 45 minutes, about 50 minutes, about 55 minutes, or about 60 minutes, or any values or ranges between above values. In one embodiment, the administration of 30 mg/mL bolus IV injection of meloxicam can provide peak analgesic effect in about 40 minutes.

Not only is the meloxicam administration as disclosed herein provide a fast onset of pain relief, it also reaches peak analgesic effect sooner than other known IV NSAIDs (Ketorolac can take 1 to 2 hours for maximum effect) and has a longer therapeutic window of at least about 24 hours (Ketorolac's duration of analgesic effect is 4 to 6 hours). See Ketorolac Tromethamine Injection Prescribing Information.

In one embodiment, a 1 mL bolus of 30 mg/mL of meloxicam provides an average plasma concentration in the range of from about 80% to about 125% of 4160±1020 ng/mL of meloxicam in a patient at about 30 minutes after intravenous administration, inclusive of all values and subranges therebetween. In one embodiment, a single 30 mg/mL bolus dose of meloxicam provides an average plasma concentration in the range of from about 2512 ng/mL to about 6475 ng/mL of meloxicam in a patient at about 30 minutes after intravenous administration, inclusive of all values and subranges therebetween. In some embodiments, a single 30 mg/mL bolus dose of meloxicam provides an average plasma concentration in the range of from about 3000 ng/mL to about 6000 ng/mL of meloxicam in a patient at about 30 minutes after intravenous administration, inclusive of all values and subranges therebetween. In other embodiments, a single 30 mg/mL bolus dose of meloxicam provides an average plasma concentration in the range of from about 3500 ng/mL to about 5500 ng/mL of meloxicam in a patient at about 30 minutes after intravenous administration, inclusive of all values and subranges therebetween. In some embodiments, a single 30 mg/mL bolus dose of meloxicam provides an average plasma concentration in the range of from about 3500 ng/mL to about 5000 ng/mL of meloxicam in a patient at about 30 minutes after intravenous administration, inclusive of all values and subranges therebetween.

In one embodiment, a 1 mL bolus of 30 mg/mL of meloxicam provides an average plasma concentration in the range of from about 80% to about 125% of 3590±708 ng/mL of meloxicam in a patient at about 60 minutes after intravenous administration, inclusive of all values and subranges therebetween. In one embodiment, a single 30 mg/mL bolus dose of meloxicam provides an average plasma concentration in the range of from about 2305 ng/mL to about 5373 ng/mL of meloxicam in a patient at about 60 minutes after intravenous administration, inclusive of all values and subranges therebetween. In some embodiments, a single 30 mg/mL bolus dose of meloxicam provides an average plasma concentration in the range of from about 2500 ng/mL to about 5000 ng/mL of meloxicam in a patient at about 60 minutes after intravenous administration, inclusive of all values and subranges therebetween. In other embodiments, a single 30 mg/mL bolus dose of meloxicam provides an average plasma concentration in the range of from about 2750 ng/mL to about 4500 ng/mL of meloxicam in a patient at about 60 minutes after intravenous administration, inclusive of all values and subranges therebetween. In some embodiments, a single 30 mg/mL bolus dose of meloxicam provides an average plasma concentration in the range of from about 3000 ng/mL to about 4000 ng/mL of meloxicam in a patient at about 60 minutes after intravenous administration, inclusive of all values and subranges therebetween.

In one embodiment, a single 30 mg/mL bolus dose of meloxicam provides an average plasma concentration in the range of from about 80% to about 125% of about 2660±394 ng/mL of meloxicam in a patient at about 120 minutes after intravenous administration, inclusive of all values and subranges therebetween. In one embodiment, a single 30 mg/mL bolus dose of meloxicam provides an average plasma concentration in the range of from about 1812 ng/mL to about 3818 ng/mL of meloxicam in a patient at about 120 minutes after intravenous administration, inclusive of all values and subranges therebetween. In some embodiments, a single 30 mg/mL bolus dose of meloxicam provides an average plasma concentration in the range of from about 1900 ng/mL to about 3800 ng/mL of meloxicam in a patient at about 120 minutes after intravenous administration, inclusive of all values and subranges therebetween. In other embodiments, a single 30 mg/mL bolus dose of meloxicam provides an average plasma concentration in the range of from about 2100 ng/mL to about 3600 ng/mL of meloxicam in a patient at about 120 minutes after intravenous administration, inclusive of all values and subranges therebetween. In some embodiments, a single 30 mg/mL bolus dose of meloxicam provides an average plasma concentration in the range of from about 2200 ng/mL to about 3400 ng/mL of meloxicam in a patient at about 120 minutes after intravenous administration, inclusive of all values and subranges therebetween.

In one embodiment, a single 30 mg/mL bolus dose of meloxicam provides an average plasma concentration in the range of from about 80% to about 125% of about 2190±262 ng/mL of meloxicam in a patient at about 4 hours after intravenous administration, inclusive of all values and subranges therebetween. In one embodiment, a single 30 mg/mL bolus dose of meloxicam provides an average plasma concentration in the range of from about 1542 ng/mL to about 3065 ng/mL of meloxicam in a patient at about 4 hours after intravenous administration, inclusive of all values and subranges therebetween. In some embodiments, a single 30 mg/mL bolus dose of meloxicam provides an average plasma concentration in the range of from about 1600 ng/mL to about 3000 ng/mL of meloxicam in a patient at about 4 hours after intravenous administration, inclusive of all values and subranges therebetween. In other embodiments, a single 30 mg/mL bolus dose of meloxicam provides an average plasma concentration in the range of from about 1800 ng/mL to about 2800 ng/mL of meloxicam in a patient at about 4 hours after intravenous administration, inclusive of all values and subranges therebetween. In some embodiments, a single 30 mg/mL bolus dose of meloxicam provides an average plasma concentration in the range of from about 1900 ng/mL to about 2600 ng/mL of meloxicam in a patient at about 4 hours after intravenous administration, inclusive of all values and subranges therebetween.

### Treatment Methods

### (i) Pre-Operative Administration of Meloxicam

Disclosed herein are methods of treating pain in a patient in need thereof, wherein the patient will be subjected to a surgical procedure, comprising administering meloxicam to the patient prior to the surgical procedure. In some embodiments, the meloxicam is administered prior to the start of the surgical procedure. As used herein, “start of surgery” or “start of surgical procedure” refers to the time of first incision. Without being bound by theory, it is thought that preoperative dosing of meloxicam provides treatment and/or management of post-surgical pain, thereby reducing the need for postoperative opioid analgesics (e.g., as evidenced by time of first opioid rescue) and improving the postoperative recovery course, as evidenced by length of stay, return of bowel function, and/or pain control satisfaction. The disclosure also provides methods of enhancing recovery from surgery and/or injury-induced traumatic wound in a patient, comprising administering meloxicam to the patient.

In some embodiments, the pre-operative administration of meloxicam is associated with, promotes and/or results in one or more of the following efficacy parameters: a decrease in the total use of opioid analgesics, an absence of the use of opioid analgesics, a decrease in summed pain intensity, an increase in the time to first use of opioid rescue medication, a decrease in pain intensity during ambulation, a decrease in the length of hospital stay, a decrease in the amount of total hospitalization charges, a decrease in the incidence of hospital readmissions, and a decrease in the requirement for skilled nursing care after discharge. In some embodiments, the one or more efficacy parameters is measured over a time period spanning from the end of surgery (H0) to Hour 24 (denoted as “H24”). In some embodiments, the one or more efficacy parameters is measured over a time period spanning from Hour (H) 0 to H48, H0 to H72, H0 to end of treatment intervals, H24 to H48, H48 to H72, H0 to H6, H6 to H12, H12 to H18, H18 to H24, H24 to H30, H30 to H36, H36 to H42, and H42 to H48, inclusive of all subranges that lie therebetween. In some embodiments, meloxicam is administered pre-operatively as part of a multimodal regimen; that is, meloxicam is administered pre-operatively in combination with the administration of one or more medications, as described below.

In some embodiments of the methods disclosed herein, meloxicam is administered for treating pain that occurs before, during, and/or after surgery, incision or wounding. In some embodiments, meloxicam may be administered prior to, during and/or after the surgery, incision and/or wound. In particular embodiments, meloxicam is administered prior to surgery. In some aspects, the pain is inflammatory, neuropathic, visceral, injury-induced pain and/or incision-induced pain. In some embodiments, the pain may be caused by severing of a nerve and/or a blood vessel. In some embodiments, the pain is chronic pain; in some embodiments, the pain is an acute pain. In some embodiments, the pain is moderate to severe pain.

In some embodiments, meloxicam is administered for treating post-surgical pain. Post-surgical pain may include two clinically important aspects, namely resting pain, or pain that occurs when the patient is not moving, and mechanical pain which is exacerbated by movement (coughing/sneezing, getting out of bed, physiotherapy, etc.). In some embodiments, resting pain is treated, in some embodiments, mechanically-induced pain (including pain resulting from movement) is treated, and in some embodiments, thermally-induced pain is treated. In some embodiments, allodynia (i.e., increased response (i.e., increased noxious sensation) to a normally non-noxious stimulus) is treated. In some embodiments, hyperalgesia (i.e., increased response to a normally noxious or unpleasant stimulus) is treated. In some embodiments, allodynia and/or hyperalgesia is thermal or mechanical (tactile) in nature, or resting pain. In some embodiments, the pain is associated with site of incision, wound or trauma, and/or proximal, at or near the site of incision, wound, and/or trauma. In some embodiments, the pain is nociceptive pain, including superficial somatic pain, deep somatic pain and visceral pain; in some embodiments, the pain is neuropathic pain such as central neuropathic pain and peripheral neuropathic pain.

In some embodiments, the surgical procedure is performed on hard and/or soft tissue. In some embodiments, the surgical procedure is performed on soft tissue. In some embodiments, the soft tissue surgery may include, but is not limited to, reproductive surgery, abdominal surgery, thoracic surgery, upper airway surgery, head and neck surgery, neurosurgery, surgical oncology and wound care and reconstruction. In some embodiments, soft tissues include, but are not limited to, tendons, ligaments, fascia, skin, fibrous tissues, fat, and synovial membranes (which are connective tissue), and muscles, nerves and blood vessels (which are not connective tissue). In other embodiments, the surgical procedure is performed on hard tissue. Hard or calcified tissues include tissues which are mineralized and have a firm intercellular matric. Non limiting examples of hard tissues are bone, tooth enamel, dentin, and cementum.

In some aspects, the surgery is open surgery, which refers to a procedure involving cutting of skin and tissues so that a surgeon has a full view of the structures or organs involved. Non-limiting examples of open surgery include removal of organs, such as gall bladder or kidneys, organ transplant, removal of a brain tumor, removal of a damaged kidney or open-heart surgery.

In some aspects, the surgery is a minimally invasive surgery, which refers to a procedure that typically does not involve generating a large incision. Non-limiting examples of minimally invasive surgery include laparoscopy, endoscopy, arthroscopy, bronchoscopy, cystoscopy, gastroscopy, hysteroscopy, laryngoscopy and sigmoidoscopy. In some embodiments, the surgical procedure is a laparoscopic surgical procedure. Typically, laparoscopy is a surgical procedure involving generating small incisions (cuts) in the wall of the abdomen and inserting a laparoscope (a thin, lighted tube) into one of the incisions. In some embodiments, during laparoscopy, other instruments may be inserted through the same or other incisions to perform procedures such as removing organs or taking tissue samples to be checked under a microscope for signs of disease. Non-limiting examples of laparoscopic procedures are gynecological surgery, lymphadenectomy, kidney surgery, radical prostatectomy, livery surgery, gallbladder removal (cholecystectomy), appendectomy, hernia repair, removal of part of the colon (colectomy) or small intestine, surgery for acid-reflux disease (fundoplication), removal of adrenal glands, and removal of the spleen. In some aspects, the surgery may be microsurgery, which typically is used for delicate work on very small body structures relying on special equipment and microscopes to magnify the area to be operated on and using tiny surgical instruments. Non-limiting examples of microsurgeries are vasectomy reversal or re-attaching a severed finger. In some embodiments, the surgery is robotic-assisted surgery, in which a surgeon maneuvers robotic arms during the procedure allowing for more precise movements. Non-limiting examples of robotic surgery include surgeries on the head and neck, gynecologic and urologic surgeries like hysterectomies and prostate cancer treatments.

In some embodiments, the surgery is colorectal surgery, while in other embodiments, the surgery is orthopedic surgery. In some embodiments, the surgery is joint replacement surgery. In some embodiments, the surgery is unilateral total knee arthroplasty.

In some embodiments, the meloxicam is administered about 5 minutes to about 24 hours, e.g., about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 1 hours 9, about 20 hours, about 21 hours, about 22 hours, about 23 hours, and about 24 hours, including all values and subranges therebetween, prior to surgical procedure. In some embodiments, the meloxicam is administered about 5 minutes to about 24 hours, including all values and subranges therebetween, prior to the start of the surgical procedure. For instance, the meloxicam may be administered about 5 minutes to about 12 hours, about 10 minutes to about 6 hours, about 20 minutes to about 3 hours, about 30 minutes to about 2 hours, about 30 minutes to about 90 minutes, about 40 minutes to about 70 minutes, about 50 minutes to about 60 minutes, including all values and subranges therebetween, prior to the start of the surgical procedure. In some embodiments, meloxicam is administered within about 24 hours, within about 12 hours, within about 6 hours, within about 5 hours, within about 4 hours, within about 3 hours, within about 2 hours, within about 1 hour, within about 45 minutes, within about 30 minutes, within about 15 minutes, within about 10 minutes, within about 5 minutes, or immediately before the start of the start of the surgical procedure. As used herein, “immediately before” means within about 2 minutes, about 1.5 minutes, about 1 minute, about 45 seconds, about 30 seconds, or about 15 seconds, including all values and ranges therein.

In some embodiments, the meloxicam is administered before the administration of anesthesia, while in other embodiments, the meloxicam is administered after the administration of anesthesia. In some embodiments, the meloxicam is administered about 2 hours to about immediately before administration of anesthesia including all values and subranges therebetween. For instance, meloxicam may be administered within about 2 hours, within about 90 minutes, within about 1 hour, within about 30 minutes, within about 15 minutes, within about 10 minutes, within about 5 minutes, including all values and subranges therebetween, or about immediately before administration of anesthesia. In some embodiments, the meloxicam is administered about 2 hours to about immediately after administration of anesthesia, including all values and subranges therebetween. For instance, the meloxicam may be administered within about 2 hours, within about 90 minutes, within about 1 hour, within about 30 minutes, within about 15 minutes, within about 10 minutes, within about 5 minutes, including all values and subranges therebetween, or about immediately after administration of anesthesia. As used herein, “immediately after” means within about 2 minutes, about 1.5 minutes, about 1 minute, about 45 seconds, about 30 seconds, or about 15 seconds, including all values and ranges therein.

In some embodiments, the patient is administered at least one dose prior to surgery. In some embodiments, the first dose of meloxicam is administered prior to surgery. In some embodiments, the patient is administered one dose prior to surgery, while in other embodiments, the patients is administered multiple doses prior to surgery. For instance, the number of doses of meloxicam that is administered to a patient prior to surgery may be 1-20 doses, 1-10 doses or 1-5 doses or as deemed appropriate by a physician. In some embodiments, the multiple doses of meloxicam that were administered to the patient prior to surgery may have been administered once a day, twice a day, three times a day, every other day, or at a frequency deemed appropriate by a physician. In some embodiments, the multiple doses of meloxicam that were administered to the patient prior to surgery may have been administered once every 12 hours, once every 18 hours, once every 24 hours, once every 36 hours, or once every 48 hours or as deemed appropriate by a physician. In some embodiments, the patient may have been on a treatment regimen that comprises administration of meloxicam over a period of time prior to surgery. The period of time prior to surgery may be 1 day, 2 days, 3 days, 4 days, 5 days, 6 days, 1 week, 2 weeks, 3 weeks, 1-11 months or a year.

In some embodiments, the methods disclosed herein further comprise administering at least one dose of meloxicam subsequent to the one or more doses of meloxicam that were administered prior to surgery. In some embodiments, the methods comprise administering meloxicam about every 18-26 hours subsequent to the dose of meloxicam that was administered prior to surgery. In some embodiments, the methods comprise administering meloxicam about every 6 hours, about every 8 hours, about every 12 hours, about every 18 hours, about every 24 hours, about every 36 hours, about every 48 hours, or at a frequency deemed appropriate by a physician, subsequent to the one or more doses of meloxicam that were administered prior to surgery. In some embodiments, the methods comprise administering meloxicam at about 18 hours, at about 24 hours, at about 36 hours, at about 48 hours, at about 54 hours, at about 72 hours, at about 96 hours, at about 5 days, at about 6 days, and so forth subsequent to the one or more doses of meloxicam that were administered prior to surgery, until required by a physician. In some embodiments, the methods disclosed herein may further comprise administering a dose of meloxicam after the completion of the surgical procedure about every 18 hours to about every 24 hours for 1 day, 2 days, 3 days, 4 days, 5 days, 6 days, 7 days, 8 days, 9 days, 10 days, 11 days, 12 days, 13 days, 14 days, or at a duration and frequency deemed appropriate by a physician.

(ii) Administration of Meloxicam in Combination with Other Medications

Disclosed herein are methods of treating pain in a patient in need thereof, comprising administering meloxicam intravenously in combination with one or more other medications. In some embodiments, the pain is a chronic pain. In some embodiments, the pain is an acute pain. In some embodiments, the pain is moderate to severe pain.

In some embodiments, the medication may be drugs used to treat pain including neuropathic pain, diabetic neuropathy, post herpetic neuralgia, central neuropathic pain or to prevent excess bleeding. In some embodiments, the medications may be analgesics, anticonvulsants, tricyclic antidepressants, anti-nausea medications or antibiotics.

In some embodiments of the methods disclosed herein, meloxicam is administered in combination with an analgesic. Analgesics are a group of drugs used to achieve relief from pain. Examples of types of analgesics include nonsteroidal anti-inflammatory drugs, opioids, medical cannabis, psychotropic agents, those that act on non-opioid pain receptors, somatostatin analogs and NMDA receptor antagonists. Examples of analgesics include acetaminophen, propyphenazone, acematacin, acetylsalicylic acid, metamizol and the salts thereof. In some embodiments, the analgesic is a short-acting analgesic. In some embodiments, the analgesic is an immediate release analgesic.

Examples of non-steroidal anti-inflammatory drugs include aspirin (acetylsalicylic acid), diflunisal (dolobid), salicylic acid and other salicylates, salsalate (disalcid), ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, diclofenac, celecoxib, aceclofenac, nabumetone, piroxicam, tenoxicam, droxicam, lornoxicam, phenylbutazone, mefenamic acid, meclofenamic acid, flufenamic acid, tolfenamic acid, etoricoxib, clonixin and licofelone.

Examples of opioid analgesics include, but are not limited to, morphine, fentanyl, sufentanil, alfentanil, hydromorphone, meperidine, methadone, buprenorphine, DADL, butorphanol, hydrocodone, oxycodone, levorphanol, dihydrocodeine, codeine, dihydromorphine, pethidine, piritramide, tilidine, tramadol, the salts thereof and related opioids.

Examples of analgesics that act on non-opioid pain receptors include alpha-2 adrenergic receptor agonists such as clonidine, tizanidine, ST-91, medetomidine, dexmedetomidine and related alpha-2 adrenergic agonists. Examples of NMDA receptor antagonists include dexmethorphan, Ifenprodil, MK-801 and related NMDA antagonists. Examples of somatostatin analogs include Octreotide, Sandostatin, Vapreotide, Lanreotide and related somatostatin analogs. Examples of other analgesics that act on non-opioid pain receptors include super oxide dismutase, baclofen, calcitonin, serotonin, vasoactive intestinal polypeptide, bombesin, omega-conopeptides and related non-opioid analgesics. Examples of medications used for neuropathic pain include pregabalin, gabapentin, duloxetine, venlafaxine, milnacipran, amitriptyline, nortriptyline, desipramine, botulinum toxin, and cannabinoids.

Examples of antibiotics include, but are not limited to, amoxicillin, doxycycline, cephalexin, ciprofloxacin, clindamycin, metronidazole, azithromycin, sulfamethoxazole/trimethoprim, amoxicillin/clavulanate and levofloxacin. In some embodiments, the antibiotic is a prophylactic antibiotic. Examples of anti-nausea medications include ondansetron, granisetron, dolasetron, dexamethasone, diphenhydramine, dimenhydrinate, lorazepam, prochlorperazine, haloperidol, metoclopramide, nabilone and palonosetron hydrochloride with netupitant, meclizine hydrochloride, emetrol, bismuth subsalicylate, promethazine, scopolamine, or a combination thereof. Examples of drugs to treat or prevent excess bleeding include tranexamic acid, oxytocin, ergotamine and carbetocin.

In some embodiments of the methods disclosed herein, meloxicam is administered prior to, during and/or after the surgery, incision and/or wound. In some embodiments, meloxicam is administered to the patient concurrently with one or more other drugs. In other embodiments, meloxicam is administered to the patient within about 24 hours, within about 12 hours, within about 6 hours, within about 5 hours, within about 4 hours, within about 3 hours, within about 2 hours, within about 1 hour, within about 45 minutes, within about 30 minutes, within about 15 minutes, within about 10 minutes, within about 5 minutes, including all values and subranges therebetween, or immediately before the administration of one or more other drugs. In some embodiments, meloxicam is administered to the patient within about 24 hours, within about 12 hours, within about 6 hours, within about 5 hours, within about 4 hours, within about 3 hours, within about 2 hours, within about 1 hour, within about 45 minutes, within about 30 minutes, within about 15 minutes, within about 10 minutes, within about 5 minutes, including all values and subranges therebetween, or immediately after the administration of one or more other drugs.

In some embodiments, the one or more drugs is administered in an amount of about 1 mg to about 1000 mg, about 1 mg to about 900 mg, about 10 mg to about 850 mg, about 100 mg to about 800 mg, about 200 mg to about 800 mg, about 200 mg to about 700 mg, about 300 mg to about 700 mg, about 400 mg to about 600 mg, about 500 mg to about 700 mg, and all the values and subranges therebetween. For instance, the one or more drugs may be administered in an amount of from about 1 mg to about 1 g, e.g., about 1 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg about 850 mg, about 900 mg about 950 mg, and about 100 mg, inclusive of all values and subranges therebetween.

In some embodiments, the one or more drugs may be administered via a systemic route or a mucosal route or a transdermal route or directly into a specific tissue. As used herein, the term “systemic administration” includes parenteral routes of administration. In particular, parenteral administration includes subcutaneous, intraperitoneal, intravenous, intraarterial, intramuscular, or intrasternal injection, intravenous, or kidney dialytic infusion techniques. As used herein, the term “mucosal administration” includes oral, intranasal, intravaginal, intra-rectal, intra-tracheal, intestinal and ophthalmic administration.

(iii) Pre-Operative Administration of Meloxicam in Combination with Other Drugs

In some embodiments, the patient is a patient who will be subjected to a surgical procedure and meloxicam is administered in combination with one or more medications before, during and/or after the surgical procedure, incision or wounding. In some embodiments, meloxicam is administered in combination with one or more medications prior to the start of, during and/or after the completion of a surgical procedure. The one or more medications may be any one or more of the drugs noted above. The surgical procedure any one of the surgical procedures noted above.

In some embodiments, the one or more drugs administered in combination with meloxicam is administered about 5 minutes to about 24 hours, including all values and subranges therebetween, prior to surgical procedure. In some embodiments, the one or more drugs is administered about 5 minutes to about 24 hours (e.g., about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 1 hours 9, about 20 hours, about 21 hours, about 22 hours, about 23 hours, and about 24 hours, including all values and subranges therebetween), prior to the start of the surgical procedure. For instance, the one or more drugs may be administered about 5 minutes to about 12 hours, about 10 minutes to about 6 hours, about 20 minutes to about 3 hours, about 30 minutes to about 2 hours, about 30 minutes to about 90 minutes or about 40 minutes to about 70 minutes, about 50 minutes to about 60 minutes, including all the values and subranges therebetween, prior to the start of the surgical procedure. In some embodiments, the one or more drugs may be administered within about 24 hours, within about 12 hours, within about 6 hours, within about 5 hours, within about 4 hours, within about 3 hours, within about 2 hours, within about 1 hour, within about 45 minutes, within about 30 minutes, within about 15 minutes, within about 10 minutes, within about 5 minutes, including all the values and subranges therebetween, or immediately before the start of the start of the surgical procedure.

In some embodiments, the one or more drugs administered in combination with meloxicam is administered before the administration of anesthesia, while in other embodiments, the one or more drugs is administered after the administration of anesthesia. In some embodiments, the one or more drugs is administered about 2 hours to about immediately before administration (e.g., within about 5 minutes, about 4 minutes, 3 minutes, 2 minutes, 1 minute) of anesthesia, including all values and subranges therebetween. For instance, one or more drugs may be administered about 2 hours, about 90 minutes, about 1 hour, about 30 minutes, about 15 minutes, about 10 minutes, about 5 minutes or immediately before administration of anesthesia. In some embodiments, the one or more drugs is administered about 2 hours to about immediately after administration of anesthesia, including all values and subranges therebetween. For instance, the one or more drugs may be administered about 2 hours, about 90 minutes, about 1 hour, about 30 minutes, about 15 minutes, about 10 minutes, about 5 minutes or immediately after administration of anesthesia.

In particular embodiments, gabapentin, acetaminophen and meloxicam are administered prior to the start of the surgical procedure; and subsequently, acetaminophen is administered every 6 hours following its pre-operative dose and IV meloxicam is administered about every 18 hours to about every 26 hours (e.g., about every 24 hours) following its pre-operative dose. In particular embodiments, gabapentin, acetaminophen and meloxicam are administered prior to the start of the surgical procedure; and subsequently, acetaminophen is administered every 8 hours following its pre-operative dose and IV meloxicam is administered about every 20 hours to about every 25 hours following its pre-operative dose.

In some embodiments, the patient is administered a dose of meloxicam prior to surgery. In some embodiments, the patient is administered at least one dose of the one or more drugs in combination with meloxicam prior to surgery. In some embodiments, the patient is administered one dose of the one or more drugs in combination with meloxicam prior to surgery, while in other embodiments, the patient is administered multiple doses of the one or more drugs in combination with one or more doses of meloxicam prior to surgery.

In some embodiments, the methods disclosed herein further comprise administering the one or more drugs, alone or in combination with meloxicam, subsequent to their pre-operative dose. In some embodiments, the one or more drugs may be administered at about every 4 hours, about every 6 hours, about every 8 hours, about every 10 hours, about every 12 hours, about every 24 hours, about every 36 hours or about every 48 hours subsequent to the dose administered prior to surgery, or at dosage and frequency as deemed appropriate by a physician.

In some embodiments, the methods disclosed herein further comprise administering at least one dose of the one or more drugs, alone or in combination with meloxicam, after the completion of the surgical procedure. For instance, the one or more drugs may be administered about 5 minutes to about 24 hours, including all values and subranges therebetween, after the completion of the surgical procedure. For instance, the one or more drugs may be administered about 5 minutes to about 12 hours, about 10 minutes to about 6 hours, about 20 minutes to about 3 hours, about 30 minutes to about 2 hours, about 30 minutes to about 90 minutes or about 40 minutes to about 70 minutes, about 50 minutes to about 60 minutes after the completion of the surgical procedure. In some embodiments, one or more drugs is administered within about 24 hours, within about 12 hours, within about 6 hours, within about 5 hours, within about 4 hours, within about 3 hours, within about 2 hours, within about 1 hour, within about 45 minutes, within about 30 minutes, within about 15 minutes, within about 10 minutes, within about 5 minutes, or immediately after the completion of the surgical procedure.

In some embodiments, meloxicam is contraindicated in patients with known hypersensitivity to meloxicam or any components of the drug product. In some embodiments, meloxicam is contraindicated in patients with a history of asthma, urticaria, or other allergic-type reactions after taking aspirin or other NSAIDs. In some embodiments, meloxicam is contraindicated in patients receiving coronary artery bypass graft (CABG) surgery. In some embodiments, meloxicam is contraindicated in patients with moderate to severe renal insufficiency who are at risk for renal failure due to volume depletion.

In some embodiments, meloxicam may cause hepatotoxicity. Accordingly, patients who are administered meloxicam may be warned about the signs and symptoms of hepatotoxicity. In some embodiments, if abnormal liver tests persist or worsen or if clinical signs and symptoms of liver disease develop, then meloxicam may be discontinued immediately. In some embodiments, meloxicam may cause hypertension. Patients taking some antihypertensive medications may have impaired response to these therapies when taking meloxicam. In some embodiments, the blood pressure of such patients may be monitored. In some embodiments, meloxicam may cause heart failure and edema. Patients with severe heart failure may avoid use of meloxicam unless benefits are expected to outweigh risk of worsening heart failure. In some embodiments, meloxicam may cause renal toxicity. In some embodiments, renal function may be monitored in patients with renal or hepatic impairment, heart failure, dehydration, or hypovolemia. Meloxicam use may be avoided in patients with advanced renal disease unless benefits are expected to outweigh risk of worsening renal function. In some embodiments, meloxicam may cause anaphylactic reactions. Emergency help may be sought if an anaphylactic reaction occurs. In some embodiments, meloxicam is contraindicated in patients with aspirin-sensitive asthma. In some embodiments, patients with preexisting asthma (without aspirin sensitivity) may be monitored for worsening of asthma. In some embodiments, meloxicam is contraindicated in patients with preexisting asthma (without aspirin sensitivity). In some embodiments, meloxicam may cause serious skin reactions and may be discontinued at first appearance of skin rash or other signs of hypersensitivity. In some embodiments, meloxicam may cause premature closure of fetal ductus arteriosus in the third trimester of pregnancy. In some embodiments, meloxicam use may be avoided in pregnant women starting 30 weeks of gestation. In some embodiments, meloxicam may cause hematologic toxicity. Hemoglobin or hematocrit may be monitored in patients with any signs or symptoms of anemia. In some embodiments, meloxicam is contraindicated in patients with any signs or symptoms of anemia. In some embodiments, meloxicam may cause nausea, headache, constipation, vomiting, pruritus or a combination thereof.

Meloxicam may interact with drugs that interfere with hemostasis (e.g., warfarin, aspirin, SSRIs/SNRIs). Patients who are concomitantly taking meloxicam with drugs that interfere with hemostasis may be monitored for bleeding. In some embodiments, if bleeding is severe, meloxicam may be terminated. In some embodiments, concomitant use of meloxicam and analgesic doses of aspirin are not recommended. In some embodiments, meloxicam may interact with ACE inhibitors, Angiotensin Receptor Blockers (ARBs), or Beta-Blockers. Concomitant use with meloxicam may diminish the antihypertensive effect of these drugs. In some embodiments, meloxicam is contraindicated with ACE inhibitors, Angiotensin Receptor Blockers (ARBs), or Beta-Blockers. In some embodiments, meloxicam interact with ACE Inhibitors and ARBs. Concomitant use with meloxicam in elderly, volume-depleted, or those with renal impairment may result in deterioration of renal function. In such high risk patients, signs of worsening renal function may be monitored. In some embodiments, meloxicam may interact with diuretics. NSAIDs may reduce the natriuretic effect of furosemide and thiazide diuretics. Patients may be monitored to assure diuretic efficacy including antihypertensive effects. In some embodiments, meloxicam is contraindicated with diuretics. In some embodiments, meloxicam may interact with digoxin. Concomitant use with meloxicam can increase serum concentration and prolong half-life of digoxin. Serum digoxin levels may be monitored. In some embodiments, meloxicam is contraindicated for use concomitant use with digoxin. In some embodiments, NSAIDs may be associated with reversible infertility. Withdrawal of meloxicam may be considered in women who have difficulties conceiving.

### EXAMPLES

### Example 1: Meloxicam 30 mg IV Injection Formulation

IV injection formulation prepared as ready-to-use formulation contains 30 mg meloxicam, povidone, sodium deoxycholate (deoxycholic acid), sucrose, and water for injection with a total volume of 1 mL in a ready-to-use vial.

Placebo contains soybean oil, egg yolk phospholipids, glycerin, fluorescein sodium, sodium folate, edetate disodium, benzyl alcohol, polysorbate 80, dextrose and water for injection. Hydrochloric acid and/or sodium hydroxide may be used for pH adjustment.

### Example 2: Safety and Efficacy of IV Meloxicam Administration Prior to Colorectal Surgery in Combination with Acetaminophen and Gabapentin

A randomized, double-blind, placebo-controlled, multicenter study evaluating the safety and efficacy of preoperative dosing with IV Meloxicam 30 mg in adult subjects undergoing open or laparoscopic colorectal surgery is being conducted. The study cohort contains approximately 50 subjects randomized (1:1) to administration of either 30 mg IV meloxicam or placebo. Adult subjects, age 18 to 80 years inclusive, scheduled to undergo colorectal surgery that is expected to result in inpatient hospitalization for at least 48-72 hours, are screened for participation at up to 20 study sites in the United States. Screening occurs within 28 days before IV meloxicam administration. After signing the informed consent, medical history, physical examination, laboratory testing, 12-lead electrocardiogram (ECG), pregnancy testing, and vital sign measurements are completed during the screening visit.

Preoperatively, the use of alvimopan is prohibited. Gabapentin 300 mg PO is administered approximately 30-90 minutes preoperatively and acetaminophen 650 mg PO or IV is administered approximately 30-90 minutes preoperatively. Midazolam 2 mg IV once is administered when needed. IV Meloxicam is administered within 30 minutes prior to the start of surgery (defined as time of first incision; Dose 1). All doses of IV Meloxicam are administered as an IV bolus over approximately 15 seconds. The start time of first dose of study drug administration (Dose 1) is used to schedule subsequent doses.

Peri-operative study protocol includes the following. No epidural medication is used. Peri-operative analgesia is maintained using IV opioids. Prohibited perioperative therapies includes NSAIDS, ketamine, transverse abdominis plane (TAP) blocks, lidocaine, and/or local instillation. Dexamethasone IV up to 4 mg and ondansetron IV 4 mg or Promethazine IV 25 mg and a scopolamine transdermal patch 0.5 mg are administered for nausea prophylaxis. Nitrous oxide, isoflurane, sevoflurane or desflurane or total intravenous anesthesia is used.

Post-operatively, subjects continue to receive IV meloxicam every 24-hours from Dose 1 so long as IV analgesia is clinically appropriate or until discharge, whichever comes first. Each subject receives at least two doses of IV Meloxicam during their participation in the study. Subjects stay at the study center for at least 48-72 hours after surgery or so long as inpatient care is clinically appropriate. A final dose of IV meloxicam is administered up to 4 hours early in subjects who are scheduled to be discharged. Subjects who did not receive a dose of IV meloxicam for >28 hours following their previous dose of study drug are considered off treatment, and did not receive further doses of IV meloxicam.

All subjects receive 650 mg of acetaminophen Q8H PO as tolerated until 24 hours following the last dose of IV meloxicam. IV morphine, or morphine patient controlled analgesia (PCA) is made available immediately postoperatively. Total morphine dose should not exceed 14 mg/hr. Patients may be supplemented with about 1 mg to about 2 mg of morphine Q1H in addition to PCA. Conversion to oral analgesia is made once subjects are tolerating liquid intake. Oral analgesia regimen is oxycodone 5 mg Q4H PO PRN, with morphine 1-4 mg IV bolus administered up to Q1H if needed for supplemental analgesia until 24 hours after the last dose of IV meloxicam. Gum chewing and use of alvimopan is prohibited. Ondansetron 4 mg IV or 8 mg PO is administered for nausea when needed.

Subjects are discharged from the study center based on their clinical status, with safety assessments performed at the earlier of 1 day (24 hours) following their last dose of IV meloxicam or at the time of discharge. Subjects are provided standard of care analgesic regimen for pain management after discharge from the study center. All treated subjects are followed through 30 days after discharge. All subjects return to the study center to complete follow-up visit 1 at 5-21 days post-discharge, with follow-up visit 2 completed by telephone 30 days post-discharge.

Safety assessments include monitoring of adverse events, wound healing assessment, vital signs, and clinical laboratory tests. Efficacy assessments include total opioid consumption, pain intensity according to an 11-point numeric pain rating scale (NPRS; 0-10) on first ambulation, time to first analgesic rescue, time to return of bowel function (including time to first flatus or bowel sounds, and first bowel movement), time to mobilization (including time to first assisted mobilization out of hospital bed and time to first independent mobilization out of hospital bed), patient global assessment of pain control, brief pain inventory, subject satisfaction with pain medication, time to hospital discharge (including time to hospital discharge order written and time to actual hospital departure), incidence of nasogastric (NG) tube insertion, length of stay, incidence of hospital readmission post initial discharge, and total cost of hospitalization.

### Example 3: Safety and Efficacy of IV Meloxicam Administration Prior to Open Unilateral Total Knee Arthroplasty in Combination with Acetaminophen and Gabapentin

Methods: A randomized, double-blind, placebo-controlled, multicenter study in adult subjects undergoing elective open unilateral total knee arthroplasty is being conducted to evaluate the safety and efficacy of administering IV meloxicam preoperatively. The surgical procedure is conducted in an inpatient hospital setting that is expected to result in a hospital stay of ≥24 hours. Each subject is screened for eligibility within 28 days before undergoing surgery on Day 1. Before surgery, approximately 200 eligible subjects are randomized in a 1:1 ratio to receive either 30 mg IV meloxicam or placebo administered as an intravenous (IV) bolus injection in ≤15 seconds.

Subjects receive oral acetaminophen 650 mg and oral gabapentin 600 mg 30 to 90 minutes before the scheduled surgical procedure. Subjects receive an appropriate prophylactic IV antibiotic, and tranexamic acid 1 gram IV 30 to 90 minutes before surgery. Following administration of intrathecal anesthesia (7.5 to 15 mg bupivacaine HCl) and before the start of surgery (i.e., time of first incision), subjects receive the first dose of IV meloxicam in less than 15 seconds according to randomization. All subjects then undergo the surgical procedure according to the investigator's clinical practice and in accordance with institutional standards.

Fentanyl and other analogues are, at times, administered intraoperatively (during the course of the surgical procedure); however, dosing with fentanyl (and other analogues) is avoided within the 30 minutes prior to the anticipated conclusion of the surgical procedure. The time and dose of fentanyl and other analogues administered are recorded. Other opioid analgesics are not administered pre- or intraoperatively, as this may confound or influence the subjects' demand for opioid analgesia during the postoperative period (Hour 0, defined as the time of last suture, staple, or steri-strip placement through hospital discharge).

Just prior to wound closure, bupivacaine HCl 0.5% 30 mL with epinephrine 0.5 mg expanded in a volume of 90 mL normal saline is injected locally into various areas of the surgical site. At the end of the surgical procedure (Hour 0) and through hospital discharge, postoperative pain management is done with IV and oral opioids according to the investigator's clinical practice and in accordance with institutional standards.

Additional doses of IV meloxicam are administered every 24 hours (±1 hour) after the first dose. Dosing is continued until the subject is either discharged from the hospital or until administration of IV meloxicam is no longer clinically appropriate. In addition to IV meloxicam, all subjects are given access to IV and/or oral (PO) opioid medication (Morphine 1-4 mg IV every 10 minutes for the first hour and then 1 to 8 mg IV Q1H PRN and oxycodone immediate release (IR) 5 mg PO Q4H (maximum of 10 mg Q4H PRN)) as needed for the management of breakthrough pain starting at Hour 0 and continuing through hospital discharge. Subjects also receive 650 mg of acetaminophen Q8H PO as tolerated until 24 hours following the last dose of meloxicam. No other analgesic agents except for IV meloxicam, acetaminophen, and the opioids designated above are permitted from Hour 0 through 48 hours. Conversion from IV to oral analgesia is made once subjects are tolerating liquid intake. Subjects remain as inpatients for at least 24 hours or until inpatient care is no longer clinically indicated. Upon discharge, subjects are provided a standard of care regimen for pain management and for physical therapy as determined by the investigator.

Twenty-four hours and 48 hours after hospital discharge qualified study staff conduct telephone interviews to assess opioid medication use, pain intensity, physical therapy visits, and utilization of healthcare resources (i.e., hospital readmission, use of skilled nursing facilities, unscheduled phone calls and/or office visits related to pain, and emergency room [ER] visits related to pain and/or other medical issues). Subjects visit the clinical site between Postoperative Days (PODs) 10 and 14 to be assessed for adverse events, wound healing, and utilization of healthcare resources. A final telephone interview is conducted on POD 30 to assess for AEs, opioid use, and utilization of healthcare resources. After the POD 30 telephone interview, subjects are discharged from the study.

Results:

This randomized, placebo-controlled study evaluated dosing with 30 mg nanocrystalline meloxicam once daily starting prior to surgery in subjects undergoing primary unilateral TKA. Subjects/investigators participating in this study were expected to follow a standardized clinical care protocol pre-, peri-, and postoperatively to maintain comparability in subject care across institutions in accordance with generally accepted good practices.

Significant reductions in postoperative opioid use were observed favoring the 30 mg nanocrystalline meloxicam treatment arm, at the primary endpoint (Hour 0 [H0] at the end of the surgical procedure, defined as the time of last suture, staple, or steri-strip placement to Hour 24 [H24]) as well as numerous secondary intervals (H48-H72, H0-H48, H0-H72, and H0 to end of treatment). During the first postsurgical day, a significantly lower total opioid dose was utilized by nanocrystalline meloxicam-treated subjects, with a 37.1% reduction vs. the placebo group (p<0.0001; 18.94 mg vs. 27.73 mg). See Table 1. This reduction in opioid requirements is of clinical importance due to continuing efforts to revise best practices reduce opioid use, particularly in acute pain settings.

TABLE 1

| | | |
|---|---|---|
| | nanocrystalline | Placebo |
| Time Interval | meloxicam 30 mg | N = 88 |

End of surgery (H0)
through Hour 24 (PSD 1)

| | | | |
|---|---|---|---|
| LS mean (SE) | 18.94 | (1.320) | 27.73 (1.371) |
| Difference (95% CI) | −8.79 | (−12.2, −5.37) | |

| | |
|---|---|
| % Difference^{b} | −31.7% |
| p-value^{a} | <0.0001 |

H0 = end of surgery;
LS = least square;
PSD = postsurgical day.
^{a}p-value from ANCOVA that included main effects of treatment and analysis center.
^{b}% difference was derived from group LS mean as 100*((nanocrystalline meloxicam/Placebo) − 1).

Mean opioid consumption was numerically lower in the nanocrystalline meloxicam 30 mg group compared with placebo at all evaluated post-surgical intervals, reaching statistically significant differences in the H48-H72, H0-H48, H0-H72, and H0 to end of treatment intervals (p<0.05). Additionally, significantly lower opioid consumption was noted after discharge in the 0-24 and 24-48 hours post discharge intervals (p<0.05). Pre- and perioperative opioid use was similar between treatment groups. A summary of total opioid use at various study intervals is provided in Table 2.

TABLE 2

| | nanocrystalline | | |
|---|---|---|---|
| Time Interval | meloxicam 30 mg | | Placebo |
| H24 through H48 (PSD 2), n | 90 | | 84 |
| LS mean (SE) | 14.37 | (1.051) | 15.91 (1.096) |
| Difference (95% CI) | −1.54 | (−4.19, 1.12) | |
| % Difference^{c} | −9.7% | | |
| p-value^{a} | 0.2549 | | |
| H48 through H72 (PSD 3), n | 65 | | 61 |
| LS mean (SE) | 6.29 | (1.148) | 9.26 (1.136) |
| Difference (95% CI) | −2.96 | (−5.65, −0.28) | |
| % Difference^{c} | −32.1% | | |
| p-value^{a} | 0.0306 | | |
| H0 through H48 (PSD 1-2), n | 93 | | 88 |
| LS mean (SE) | 33.28 | (1.931) | 43.99 (2.007) |
| Difference (95% CI) | −10.7 | (−15.7, −5.70) | |
| % Difference^{c} | −24.3% | | |
| p-value^{a} | <0.0001 | | |
| H0 through H72 (PSD 1-3), n | 93 | | 88 |
| LS mean (SE) | 39.45 | (2.338) | 52.63 (2.429) |
| Difference (95% CI) | −13.2 | (−19.2, −7.12) | |
| % Difference^{c} | −25.0% | | |
| p-value^{a} | <0.0001 | | |
| H0 through EOT, n | 93 | | 88 |
| LS mean (SE) | 32.24 | (2.271) | 43.50 (2.360) |
| Difference (95% CI) | −11.3 | (−17.1, −5.36) | |
| % Difference^{c} | −25.9% | | |
| p-value^{a} | 0.0002 | | |
| Hospital discharge through | 92 | | 85 |
| 24-hour telephone interview, n | | | |
| LS mean (SE) | 6.23 | (0.540) | 7.70 (0.562) |
| Difference (95% CI) | −1.47 | (−2.87, −0.07) | |
| % Difference^{c} | −19.1% | | |
| p-value^{a} | 0.0394 | | |
| 24-hour interview through | 89 | | 85 |
| 48-hour telephone interview, n | | | |
| LS mean (SE) | 5.34 | (0.502) | 7.02 (0.515) |
| Difference (95% CI) | −1.68 | (−2.96, −0.40) | |

| | |
|---|---|
| % Difference^{c} | −23.9% |
| p-value^{a} | 0.0105 |

EOT = end of treatment (earlier of discharge or last study dose (LSD) + 1);
H0 = end of surgery;
LS = least square;
PSD = post surgery day.
^{a}p-value from ANCOVA that included main effects of treatment and analysis center.
b Excludes Dose 2.
^{c}% difference was derived from group LS mean as 100*((nanocrystalline meloxicam/Placebo) − 1).

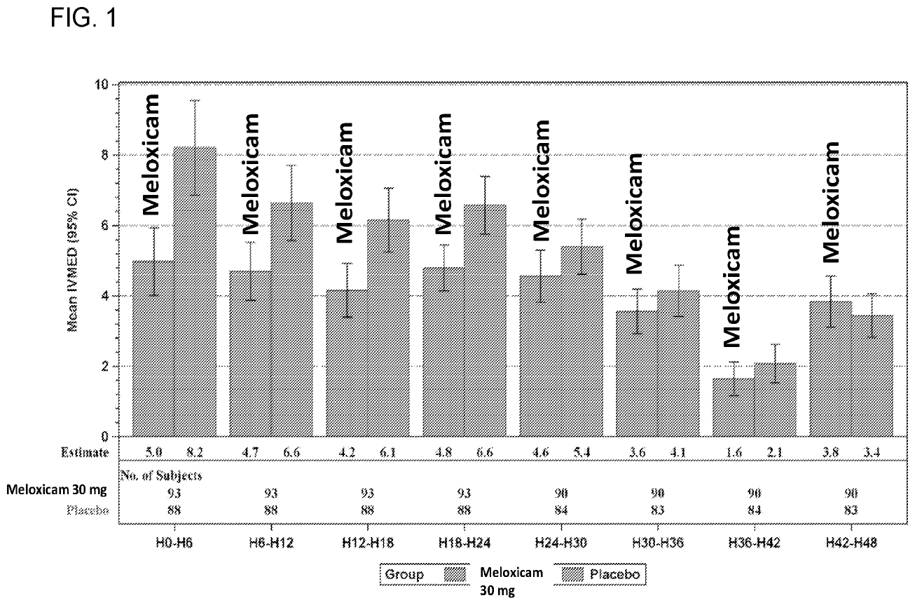

An additional unplanned analysis (ad-hoc) was performed to evaluate the use of opioids at 6-hour intervals following the end of surgery. Opioid use was numerically lower in the nanocrystalline meloxicam 30 mg group in every 6-hour interval except H42-H48. Significant reductions were observed in each 6-hour interval through H24 demonstrating a significant reduction in opioid use for nanocrystalline meloxicam 30 mg vs. placebo. A summary of opioid use at 6-hour intervals following end of surgery is provided in Table 3 and FIG. 1.

TABLE 3

| | nanocrystalline | | Placebo |
|---|---|---|---|
| Time Interval | meloxicam 30 mg | | (N = 88) |
| H0-H6, LS mean (95% CI) | 5.10 | (3.88, 6.31) | 8.27 (7.01, 9.53) |
| Difference (95% CI) | −3.17 | (−4.77, −1.58) | |
| % Difference^{a} | −38.3% | | |
| p-value | 0.0001 | | |
| H6-H12, LS mean (95% CI) | 4.70 | (3.76, 5.64) | 6.68 (5.70, 7.65) |
| Difference (95% CI) | −1.98 | (−3.21, −0.75) | |
| % Difference^{a} | −29.6% | | |
| p-value | 0.0018 | | |
| H12-H18, LS mean (95% CI) | 4.23 | (3.38, 5.07) | 6.12 (5.24, 7.00) |
| Difference (95% CI) | −1.89 | (−3.00, −0.78) | |
| % Difference^{a} | −30.9% | | |
| p-value | 0.0010 | | |
| H18-H24, LS mean (95% CI) | 4.92 | (4.14, 5.70) | 6.67 (5.85, 7.48) |
| Difference (95% CI) | −1.75 | (−2.77, −0.72) | |
| % Difference^{a} | −26.2% | | |
| p-value | 0.0009 | | |
| H24-H30, LS mean (95% CI) | 4.52 | (3.74, 5.31) | 5.32 (4.51, 6.14) |
| Difference (95% CI) | −0.80 | (−1.80, 0.20) | |
| % Difference^{a} | −15.0% | | |
| p-value | 0.1166 | | |
| H30-H36, LS mean (95% CI) | 3.68 | (2.95, 4.42) | 4.31 (3.53, 5.08) |
| Difference (95% CI) | −0.62 | (−1.57, 0.33) | |
| % Difference^{a} | −14.6% | | |
| p-value | 0.1964 | | |
| H36-H42, LS mean (95% CI) | 1.70 | (1.16, 2.25) | 2.14 (1.57, 2.70) |
| Difference (95% CI) | −0.43 | (−1.13, 0.26) | |
| % Difference^{a} | −20.6% | | |
| p-value | 0.2205 | | |
| H42-H48, LS mean (95% CI) | 3.72 | (3.01, 4.43) | 3.39 (2.65, 4.14) |
| Difference (95% CI) | 0.33 | (−0.58, 1.24) | |

| | |
|---|---|
| % Difference^{a} | 9.7% |
| p-value | 0.4806 |

LS = least square
^{a}% difference was derived from group LS mean as 100*((nanocrystalline meloxicam/Placebo) − 1).

In addition to reduced opioid consumption, significant reductions in the summed pain intensity (SPI) were observed in the 30 mg nanocrystalline meloxicam treatment group at multiple intervals. It is notable that no imputations or adjustments were made to pain intensity (PI) scores to account for opioid rescue use, thus these significant reductions in SPI came in spite of the trend for higher opioid use (significant at multiple intervals) in the placebo group.

A total of 14 pain intensity (PI) scores were scheduled: 13 PI scores were collected within the first 48 hours after end of surgery plus a PI score at the time of hospital discharge. The time points for scheduled PI scores included: Upon arrival at the post anesthesia care unit (PACU); Time points relative to first dose of study drug (Time 0): 4 hours±15 minutes, 6 hours±15 minutes, 8 hours±30 minutes, 10 hours±1 hour, 12 hours±1 hour, 16±1 hour, 20 hours±1 hour, 24 hours±1 hour (before study drug administration, if indicated), 30 hours±2 hours, 36 hours±2 hours, 42 hours±2 hours, and 48 hours±2 hours (before study drug administration, if indicated), when the subject is awake; and before hospital discharge. In addition, unscheduled PI assessments were performed prior to use of rescue analgesic; PI was also assessed prior to each ambulation attempt (Table 6), and the worst pain intensity during an ambulation was collected; however, the worst pain scores during ambulation were not included in the SPI analysis.

During the 24-hour period following the first study dose (T0), the summed PI (SPI₂₄) was significantly lower in nanocrystalline meloxicam treated subjects compared with placebo (p<0.0001). See Table 4.

TABLE 4

| | | |
|---|---|---|
| | nanocrystalline | |
| | meloxicam 30 mg | Placebo |
| Parameter | N = 93 | N = 88 |

SPI₂₄

| | | | |
|---|---|---|---|
| LS mean (SE) | 5328 | (238.1) | 6854 (248.6) |
| Difference (95% CI) | −1525 | (−2148, −903) | |

| | |
|---|---|
| p-value^{a} | <0.0001 |

A summary of SPI₂₄ data is provided in Table 4, with analysis of additional SPI intervals summarized below. Significant reductions in SPI were observed in the time from first study dose (T0) to the first assisted ambulation and to the time of discharge in the nanocrystalline meloxicam 30 mg group compared with placebo (p≤0.0235). SPI scores for T0 to the first independent ambulation were numerically lower for nanocrystalline meloxicam, but did not reach significance. See Table 5.

TABLE 5

| | | |
|---|---|---|
| | nanocrystalline | |
| | meloxicam 30 mg | Placebo |
| Parameter | N = 93 | N = 88 |

| SPI_{T0-AA} | | | |
|---|---|---|---|
| Number of subjects | 91 | | 78 |
| LS mean (SE) | 2211 | (355.4) | 3307 (378.6) |
| Difference (95% CI) | −1096 | (−2043, −149) | |
| p-value^{a} | 0.0235 | | |
| SPI_{T0-IA} | | | |
| Number of subjects | 17 | | 11 |
| LS mean (SE) | 5170 | (1566) | 8151 (2262) |
| Difference (95% CI) | −2980 | (−8885, 2924) | |
| p-value^{a} | 0.3018 | | |
| SPI_{T0-DC} | | | |
| Number of subjects | 93 | | 84 |
| LS mean (SE) | 10541 | (993.0) | 15670 (1045) |
| Difference (95% CI) | −5129 | (−7734, −2523) | |

| | |
|---|---|
| p-value^{a} | 0.0001 |

AA = time of first assisted ambulation;
ANCOVA = analysis of covariance;
DC = hospital discharge;
IA = time of first unassisted/independent ambulation;
LS = least square;
SE = standard error;
T0 = time of first dose
^{a}p-value from an ANCOVA that included treatment and investigator center.

In addition to the scheduled PI assessments performed at various intervals as described above, PI was assessed prior to ambulation, and a worst PI during ambulation was collected after completion the ambulation session. No significant differences in PI were observed prior to or during the first assisted and/or independent ambulation in the study. Mean PI scores were numerically lower for nanocrystalline meloxicam prior to ambulation for first assisted and independent ambulation compared to placebo. A summary of PI scores on first ambulation is provided in Table 6.

TABLE 6

| | | |
|---|---|---|
| | nanocrystalline | |
| | meloxicam 30 mg | Placebo |
| Parameter | N = 93 | N = 88 |

Pain Intensity on First Assisted Ambulation

| Pain intensity prior to | 91 | | 78 |
|---|---|---|---|
| ambulation, n | | | |
| LS mean (SE) | 4.01 | (0.281) | 4.67 (0.299) |
| Difference (95% CI) | −0.66 | (−1.41, 0.08) | |
| p-value^{a} | 0.0818 | | |
| Worst pain intensity | 90 | | 75 |
| during ambulation, n | | | |
| LS mean (SE) | 5.82 | (0.268) | 5.81 (0.287) |
| Difference (95% CI) | 0.02 | (−0.69, 0.72) | |

| | |
|---|---|
| p-value^{a} | 0.9647 |

Pain Intensity on First Unassisted/Independent Ambulation

| Pain intensity prior to | 17 | | 10 |
|---|---|---|---|
| ambulation, n | | | |
| LS mean (SE) | 1.97 | (0.612) | 3.72 (0.917) |
| Difference (95% CI) | −1.74 | (−4.11, 0.63) | |
| p-value^{a} | 0.1395 | | |
| Worst pain intensity | 17 | | 11 |
| during ambulation, n | | | |
| LS mean (SE) | 3.89 | (0.654) | 5.56 (0.945) |
| Difference (95% CI) | −1.67 | (−4.14, 0.80) | |

| | |
|---|---|
| p-value^{a} | 0.1713 |

Pain intensity scale: 0 = no pain to 10 = worst pain
^{a}p-value from an ANOVA that included treatment and investigator center.

Time to first ambulation was analyzed by use of first assisted and first independent ambulations following surgery. The Kaplan-Meier (KM) 50% and mean times to first assisted ambulation were numerically shorter in the nanocrystalline meloxicam treatment group, but failed to reach significance (p=0.0512). No differences were apparent in the time to first independent ambulation, given the fact that very few subjects (18% in the nanocrystalline meloxicam group and 11% in the Placebo group) were able to ambulate independently while hospitalized. A summary of time to first ambulation is provided in Table 7.

TABLE 7

| | | |
|---|---|---|
| | nanocrystalline | |
| | meloxicam 30 mg | Placebo |
| Parameter | N = 93 | N = 88 |

Time (hr) from end of surgery (H0)
to first assisted ambulation:

| Number (%) subjects with | 91 | (97.8) | 78 | (88.6) |
|---|---|---|---|---|
| assisted ambulation | | | | |
| KM 25^{th} percentile (95% CI) | 4.27 | (3.87, 4.95) | 4.48 | (3.83, 5.33) |
| KM 50^{th} percentile (95% CI) | 6.92 | (5.65, 8.05) | 10.08 | (5.83, 16.50) |
| KM 75^{th} percentile (95% CI) | 18.95 | (9.88, 22.78) | 22.52 | (17.98, 26.90) |
| KM Mean (SE) | 12.55 | (1.40) | 15.66 | (1.57)^{b} |
| Hazards ratio (95% CI) | 1.361 | (0.998, 1.855) | | |
| p-value^{a} | 0.0512 | | | |
| Time (hr) from end of surgery (H0) | | | | |
| to unassisted/independent ambulation: | | | | |
| Number (%) subjects with | 17 | (18.3) | 11 | (12.5) |
| unassisted/independent ambulation | | | | |
| KM 25^{th} percentile (95% CI) | 70.85 | (44.93, NA) | NA | (49.17, NA) |
| KM 50^{th} percentile (95% CI) | NA | (71.08, NA) | NA | (NA, NA) |
| KM 75^{th} percentile (95% CI) | NA | (NA, NA) | NA | (NA, NA) |
| KM Mean (SE) | 63.94 | (1.78)^{b} | 50.00 | (0.95)^{b} |

| | | |
|---|---|---|
| Hazards ratio (95% CI) | 1.563 | (0.723, 3.378) |

| | |
|---|---|
| p-value^{a} | 0.2558 |

H0 = end of surgery;
KM = Kaplan-Meier.
^{a}p-value from Cox proportional hazards analysis model. Cox PH model compared the probability of having the event in nanocrystalline meloxicam treated subjects with that in placebo treated subjects.
^{b}KM mean (SE) time (hr) to event could be underestimated because the largest observation was censored and the estimation was restricted to the largest event time.

Further, Patient Global Assessment (PGA) and Overall Benefit of Analgesia Score (OBAS) assessments (see below) saw early significant reductions favoring nanocrystalline meloxicam, and maintained a favorable response for nanocrystalline meloxicam treated subjects at subsequent time points. These findings support that subjects are having improved pain control and satisfaction compared to that seen with a traditional care regimen (placebo+opioid rescue), while reducing opioid requirements in subjects, demonstrating the benefits of a multimodal pain regimen in this setting that includes 30 mg nanocrystalline meloxicam once daily. As part of multimodal pain management, subjects received oral acetaminophen 650 mg and oral gabapentin 600 mg 30 to 90 minutes before the scheduled surgical procedure. Subjects continued to receive acetaminophen 650 mg PO Q8H through last study dose+1 day (LSD+1). Subjects also received an appropriate prophylactic IV antibiotic and tranexamic acid 1 gram IV 30 to 90 minutes before surgery.

Starting on post-operative day (POD 1) and continuing each day through hospital discharge or last study dose (LSD)+1, whichever occurred first, study staff asked subjects to evaluate their pain control during the preceding period according to the following scale: 0-poor, 1-fair, 2-good, 3-very good, or 4-excellent”. Subject reported PGA scores were grouped as positive (rated 2-Good or above) or not positive (rated 1-Fair or below) for analysis. On POD1, significantly more subjects in the nanocrystalline meloxicam treatment group reported positive assessments on the PGA compared with placebo (80.4% vs. 63.1%; p=0.0105). Similarly, numerically more subjects in the nanocrystalline meloxicam treatment group reported positive assessment on POD2 and prior to discharge, but the differences were not statistically significant. Because most of subjects were discharged before POD 3, the number of subjects available for this assessment on POD3 and POD4 was very small. A summary of the incidence of positive PGA assessments is provided in Table 8.

TABLE 8

| | nanocrystalline | | |
|---|---|---|---|
| | meloxicam 30 mg | Placebo | p-value^{a} |
| | Number of subjects with positive ratings^{b}/ | | |
| Parameter | Total number of subjects with assessment (%) | | |
| POD 1 | 74/92 (80.4) | 53/84 (63.1) | 0.0105 |
| POD 2 | 70/76 (92.1) | 57/66 (86.4) | 0.2698 |
| POD 3 | 11/12 (91.7) | 20/21 (95.2) | 0.6536 |
| POD 4 | 0/0 | 1/1 (100) | N/A |
| Prior to discharge | 86/92 (93.5%) | 74/84 | 0.2618 |

POD = postoperative day
^{a}p-value from Cochran-Mantel-Haenszel test controlling for analysis center.
^{b}Positive ratings include good (2), very good (3), and excellent (4).

Starting on POD 1 and continuing each day through hospital discharge or last study dose (LSD)+1, whichever occurs first, subjects to responded to the 7 items contained in the OBAS questionnaire (as described in Lehmann 2010, incorporated herein by reference in its entirety for all purposes) as follows: 1. Current pain at rest on a scale between 0=minimal pain and 4=maximum imaginable pain; 2. Distress and bother from vomiting in the past 24 hours (0=not at all to 4=very much); 3. Distress and bother from itching in the past 24 hours (0=not at all to 4=very much); 4. Distress and bother from sweating in the past 24 hours (0=not at all to 4=very much); 5. Distress and bother from freezing in the past 24 hours (0=not at all to 4=very much); 6. Distress and bother from dizziness in the past 24 hours (0=not at all to 4=very much); and 7. Satisfaction with pain treatment during the past 24 hours (0=not at all to 4=very much).

The two primary scores resulting from the OBAS assessment is the overall OBAS score as well as the Opioid Dimension Distress Score (ODDS). The OBAS was significantly lower for nanocrystalline meloxicam on POD1 compared with placebo (p=0.0027), and remained numerically lower on subsequent days, but failed to reach significance. The ODDS was numerically lower for nanocrystalline meloxicam on POD1, POD2, and prior to discharge, but was lower for placebo on POD3. None of the differences in ODDS results were significantly different. A summary of OBAS and ODDS scoring results is provided in Table 9.

TABLE 9

| | | |
|---|---|---|
| | nanocrystalline | |
| Parameter | meloxicam 30 mg | Placebo |

Overall Benefit of Analgesia Score (OBAS)

| POD1, n | 92 | | 84 |
|---|---|---|---|
| LS mean (SE) | 4.45 | (0.360) | 5.90 (0.375) |
| Difference (95% CI) | −1.45 | (−2.39, −0.51) | |
| p-value^{a} | 0.0027 | | |
| POD2, n | 76 | | 65 |
| LS mean (SE) | 3.85 | (0.379) | 4.49 (0.400) |
| Difference (95% CI) | −0.64 | (−1.57, 0.30) | |
| p-value^{a} | 0.1803 | | |
| POD3, n | 12 | | 21 |
| LS mean (SE) | 3.89 | (0.805) | 3.95 (0.705) |
| Difference (95% CI) | −0.05 | (−1.83, 1.73) | |
| p-value^{a} | 0.9524 | | |
| Prior to Discharge, n | 92 | | 84 |
| LS mean (SE) | 3.82 | (0.322) | 4.51 (0.336) |
| Difference (95% CI) | −0.69 | (−1.53, 0.15) | |

| | |
|---|---|
| p-value^{a} | 0.1054 |

Opioid Dimension Distress Score (ODDS)

| POD1, n | 92 | | 84 |
|---|---|---|---|
| LS mean (SE) | 1.89 | (0.260) | 2.48 (0.271) |
| Difference (95% CI) | −0.59 | (−1.27, 0.09) | |
| p-value^{a} | 0.0872 | | |
| POD2, n | 76 | | 66 |
| LS mean (SE) | 1.59 | (0.294) | 1.90 (0.309) |
| Difference (95% CI) | −0.32 | (−1.04, 0.40) | |
| p-value^{a} | 0.3870 | | |
| POD3, n | 12 | | 21 |
| LS mean (SE) | 2.07 | (0.709) | 1.73 (0.621) |
| Difference (95% CI) | 0.34 | (−1.23, 1.91) | |
| p-value^{a} | 0.6564 | | |
| Prior to Discharge, n | 92 | | 84 |
| LS mean (SE) | 1.70 | (0.255) | 1.86 (0.266) |
| Difference (95% CI) | −0.16 | (−0.83, 0.51) | |

| | |
|---|---|
| p-value^{a} | 0.6357 |

ANCOVA = analysis of covariance;
POD = postoperative day;
LS = least square;
SE = standard error
^{a}p-value from an ANCOVA that included treatment and investigator center.

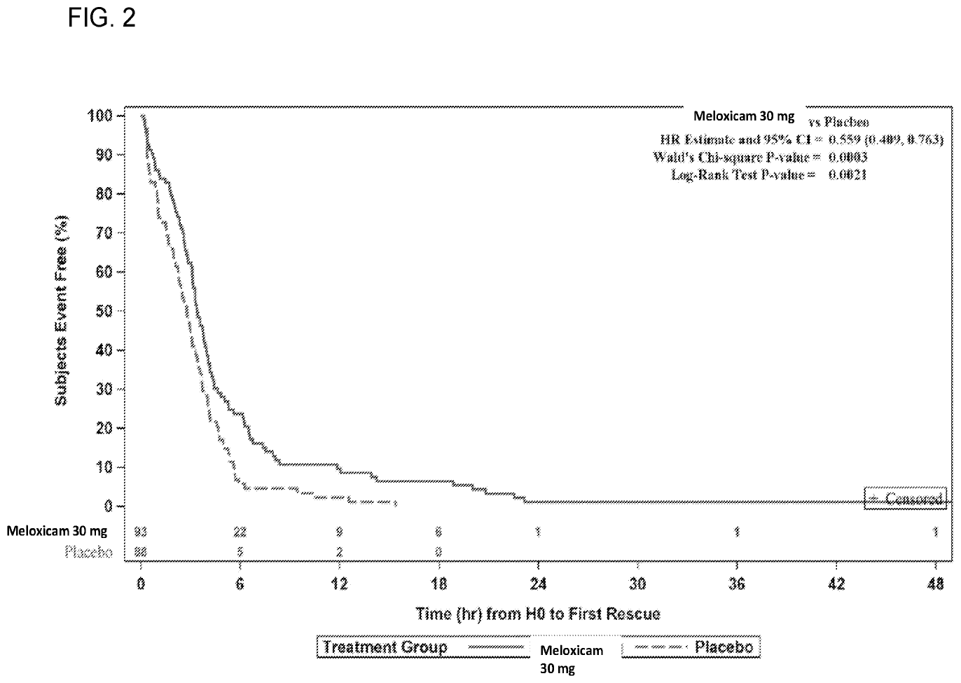

In addition to the significant reductions in opioid use, this study demonstrated a significant delay to the time of first opioid rescue. Following the end of surgery subjects were able to request opioid analgesia as needed for pain symptoms. Subjects treated with nanocrystalline meloxicam 30 mg had a significantly longer time from end of surgery to first opioid requirement (via IV or oral administration) compared to placebo subjects (p=0.0003). A summary of time from end of surgery to first opioid rescue is provided in Table 10, and a Kaplan-Meier survival curve plot for the time to first opioid rescue (IV or oral), is provided in FIG. 2.

TABLE 10

| | | |
|---|---|---|
| | nanocrystalline | |
| | meloxicam 30 mg | Placebo |
| Parameter | N = 93 | N = 88 |

Time (hr) from end of surgery
(H0) to first rescue (IV or oral):

| Subjects (%) censored | 1 | (1.1) | 0 | |
|---|---|---|---|---|
| KM 25^{th} percentile (95% CI) | 2.20 | (1.13, 2.65) | 1.06 | (0.57, 1.80) |
| KM 50^{th} percentile (95% CI) | 3.38 | (3.10, 3.97) | 2.78 | (2.23, 3.28) |
| KM 75^{th} percentile (95% CI) | 5.30 | (4.17, 6.77) | 4.08 | (3.57, 4.97) |
| KM Mean (SE) | 4.94 | (0.54)^{b} | 3.09 | (0.28) |

| | | |
|---|---|---|
| Hazard ratio (95% CI) | 0.559 | (0.409, 0.763) |

| | |
|---|---|
| p-value^{a} | 0.0003 |

H0 = end of surgery,
KM = Kaplan-Meier
^{a}p-value from Cox proportional hazards analysis model. Cox PH model compared the probability of having the event in nanocrystalline meloxicam treated subjects with that in placebo treated subjects.
^{b}KM mean (SE) time (hr) to event could be underestimated because the largest observation was censored and the estimation was restricted to the largest event time.

Additional time to rescue analysis is provided below. As seen with the time to first opioid rescue (IV or oral) described above, a significant delay to first opioid use was seen via the individual rescue routes of administration as well. The time to first IV and first oral opioid rescue in subjects were significantly longer in subjects receiving nanocrystalline meloxicam 30 mg compared with placebo (p≤0.0038). A summary of time to first IV and first oral opioid rescue is provided in Table 11.

TABLE 11

| | | |
|---|---|---|
| | nanocrystalline | |
| | meloxicam 30 mg | Placebo |
| Parameter | N = 93 | N = 88 |

Time from end of surgery
(H0) to first IV rescue, (hr):

| | | | | |
|---|---|---|---|---|
| Subjects (%) censored | 30 | (32.3) | 11 | (12.5) |
| KM 25^{th} percentile (95% CI) | 2.65 | (1.47, 4.42) | 1.11 | (0.57, 1.97) |
| KM 50^{th} percentile (95% CI) | 6.22 | (4.83, 8.15) | 3.65 | (2.95, 5.53) |
| KM 75^{th} percentile (95% CI) | 18.25 | (9.40, 24.70) | 6.77 | (5.90, 10.88) |
| KM Mean (SE) | 10.85 | (1.38)^{b} | 6.16 | (0.83) |

| | | |
|---|---|---|
| Hazard ratio (95% CI) | 0.555 | (0.393, 0.786) |

| | |
|---|---|
| p-value^{a} | 0.0009 |
| Time from end of surgery | |
| (H0) to first oral rescue, (hr): | |

| Subjects (%) censored | 1 | (1.1) | 0 | |
|---|---|---|---|---|
| KM 25^{th} percentile (95% CI) | 3.28 | (2.75, 3.75) | 2.70 | (2.40, 3.07) |
| KM 50^{th} percentile (95% CI) | 4.28 | (3.88, 5.60) | 3.94 | (3.30, 4.38) |
| KM 75^{th} percentile (95% CI) | 8.12 | (6.52, 12.05) | 5.28 | (4.68, 6.17) |
| KM Mean (SE) | 7.69 | (0.85)^{b} | 5.22 | (0.52) |

| | | |
|---|---|---|
| Hazard ratio (95% CI) | 0.636 | (0.469, 0.863) |

| | |
|---|---|
| p-value^{a} | 0.0036 |

H0 = end of surgery;
KM = Kaplan-Meier.
^{a}p-value from Cox proportional hazards analysis model. Cox PH model compared the probability of having the event in nanocrystalline meloxicam treated subjects with that in placebo treated subjects.
^{b}KM mean (SE) time (hr) to event could be underestimated because the largest observation was censored and the estimation was restricted to the largest event time.

It was notable that the number of opioid free subjects did increase over time, with greater improvement in the nanocrystalline meloxicam treatment group. While the current study sought to observe the incidence of opioid free subjects, the care protocol made no efforts to specifically reduce or avoid the use of opioid medications, with opioids identified as the primary rescue medication. Notably, significant reductions in opioid use during the inpatient period were observed to continue in the first 48 hour period following discharge.

These findings further support the beneficial role of nanocrystalline meloxicam used as part of a multimodal analgesic regimen in an acute pain setting. Multimodal regimens, those containing 2 or more analgesics with differing mechanisms of action, have consistently demonstrated the benefits of a diversified analgesic regiment to provide improved pain relief and reducing patient opioid requirements. Components of a multimodal regimen may include analgesics (including opioids, NSAIDs, acetaminophen, gabapentinoids, and/or serotonergic agents), local or regional nerve blocks, and/or intraarticular and wound infiltrations among other components.

Additionally, hospital charge data suggest that nanocrystalline meloxicam had less healthcare resource use and reduced healthcare costs compared to placebo. Length of hospital stay was measured as the time (hours) from the end of surgery (H0) to the time the discharge order was written, and the time of actual hospital discharge. No significant difference was identified between groups in the length of hospital stay, though Kaplan-Meier (KM) 50ₜₕ percentiles and KM Mean values were numerically lower for nanocrystalline meloxicam compared with placebo. A summary of length of hospital stay is provided in Table 12.

TABLE 12

| | | |
|---|---|---|
| | nanocrystalline | |
| | meloxicam 30 mg | Placebo |
| Parameter | N = 93 | N = 88 |

Time from H0 to hospital
discharge order written (hours):

| KM 25^{th} percentile (95% CI) | 39.25 | (25.47, 42.67) | 36.28 | (23.22, 42.30) |
|---|---|---|---|---|
| KM 50^{th} percentile (95% CI) | 45.83 | (44.17, 47.68) | 47.07 | (43.87, 49.33) |
| KM 75^{th} percentile (95% CI) | 50.40 | (48.57, 68.75) | 64.87 | (50.73, 69.03) |
| KM Mean (SE) | 46.09 | (1.749) | 49.93 | (3.119) |
| Hazards ratio (95% CI) | 1.113 | (0.819, 1.515) | | |
| p-value^{a} | 0.4935 | | | |
| Time from H0 to actual | | | | |
| hospital discharge (hours): | | | | |
| KM 25^{th} percentile (95% CI) | 46.30 | (29.55, 48.50) | 46.89 | (31.90, 48.53) |
| KM 50^{th} percentile (95% CI) | 51.40 | (49.35, 52.62) | 51.74 | (49.52, 53.80) |
| KM 75^{th} percentile (95% CI) | 54.68 | (53.17, 73.80) | 73.58 | (54.82, 75.78) |
| KM Mean (SE) | 52.10 | (1.768) | 56.92 | (3.094) |

| | | |
|---|---|---|
| Hazards ratio (95% CI) | 1.147 | (0.841, 1.566) |

| | |
|---|---|
| p-value^{a} | 0.3869 |

^{a}p-value from Cox proportional hazards analysis model. Cox PH model compared the probability of having the event in nanocrystalline meloxicam treated subjects with that in placebo treated subjects

Charges for the initial hospitalization were determined using UB-04 and/or similar hospital claim forms used for billing purposes. Total hospital charges, as recorded on the UB-04/hospital claims forms, were recorded. In addition, total hospital charges were determined from the billing codes and/or procedure codes for all hospital care from the time of hospital admission until discharge as reported on the UB-04/hospital claims forms. Mean hospitalization charges were lower in the nanocrystalline meloxicam 30 mg treatment group compared with placebo, with an apparent 10.2% reduction in charges. A summary of total hospitalization charges is provided in Table 13.

TABLE 13

| | nanocrystalline | | | |
|---|---|---|---|---|
| | meloxicam 30 mg | | Placebo | |
| Parameter | N = 93 | | N = 88 | |
| Total cost of hospitalization (dollars) | | | | |
| Mean (SD) | 56424.13 | (29925.32) | 62864.14 | (45254.42) |
| Median (Min, Max) | 49270.50 | (26302.8, 161022.3) | 50506.54 | (12962.6, 255070.0) |

Qualified study staff conducted telephone interviews 24-hours and 48-hours after hospital discharge, POD 10-14 visit, and on POD 30. During each phone interview, subjects were asked about opioid medication use, pain intensity, physical therapy visits, and utilization of healthcare resources (i.e., hospital readmission, use of skilled nursing facilities, unscheduled phone calls and/or office visits related to pain, and emergency room [ER] visits related to pain). Overall, nanocrystalline meloxicam treated subjects had a lower incidence of hospital readmissions (1.1% vs. 3.4%), ER visits due to pain (0 vs. 4.5%), and doctor calls due to pain (4.3% vs. 10.2%) compared with placebo. No subjects reported unscheduled doctor visits due to pain. A summary of hospital readmissions and ER visits, doctor visits and doctor calls due to pain is provided in Table 14.

TABLE 14

| | nanocrystalline | | Placebo | |
|---|---|---|---|---|
| | meloxicam 30 mg | | N = 88 | |
| Parameter | n (%) | Events | n (%) | Events |
| All cause hospital readmissions | 1 (1.1) | 1 | 3 (3.4) | 3 |
| ER visits due to pain | 0 | 0 | 4 (4.5) | 4 |
| Unscheduled doctor visits due | 0 | 0 | 0 | 0 |
| to pain | | | | |
| Phone calls due to pain | 4 (4.3) | 4 | 9 (10.2) | 9 |

ER = emergency room

During each post-hospital follow-up phone interview and during the POD 10-14 visit, subjects were asked about utilization of healthcare resources including the use of skilled nursing facilities. There was a numerically greater incidence of subjects requiring skilled nursing care after discharge in the placebo group compared with nanocrystalline meloxicam with 14.8% vs. 5.4% of subjects requiring additional care. A summary of skilled nursing care use is provided in Table 15.

TABLE 15

| | | |
|---|---|---|
| | nanocrystalline | Placebo |
| Parameter | meloxicam 30 mg | N = 88 |

| | | | | |
|---|---|---|---|---|
| Subjects requiring skilled | 5 | (5.4) | 13 | (14.8) |
| nursing facility care after | | | | |

| | | |
|---|---|---|
| Total time spent in a skilled | | |
| n | 93 | 88 |

| | | | | |
|---|---|---|---|---|
| Mean (SD) | 0.6 | (3.12) | 1.9 | (4.99) |
| Median (Min, Max) | 0 | (0.0, 22) | 0 | (0.0, 22) |

Overall, these results demonstrate a significant benefit of preoperative administration of nanocrystalline meloxicam, over placebo. These results also support that administering nanocrystalline meloxicam preoperatively, as part of a multimodal regimen (for example, in combination with acetaminophen and gabapentin) has superior effects in comparison to administering placebo.

Subjects in the nanocrystalline meloxicam 30 mg treatment group had significantly lower opioid consumption during the first postsurgical day (end of surgery through 24 hours after surgery; primary efficacy endpoint) with a 31.7% reduction vs. placebo (p<0.0001). Subjects in the nanocrystalline meloxicam 30 mg treatment group also had a significantly lower summed pain intensity (SPI) on the first postsurgical day and throughout their inpatient course (p<0.0001); no imputations in PI were made to account for opioid use, thus placebo SPI scores include response from significantly higher opioid use during multiple assessment intervals. Despite these differences, the majority of subjects received at least one opioid medication on the first postsurgical day, with no difference in the incidence of opioid free subjects between treatments. However, subjects who received preoperative nanocrystalline meloxicam 30 mg did have a significantly longer time to first opioid rescue after surgery compared with placebo (p=0.0021). Significant reductions in opioid use were also seen throughout inpatient treatment (p=0.0002) and in the study overall (p=0.0073). A significant difference was noted on Day 1 for PGA and OBAS scores favoring nanocrystalline meloxicam (p=0.0105 and 0.0027). Subjects in the nanocrystalline meloxicam 30 mg treatment group also utilized fewer healthcare resources after discharge, including numerically lower incidences of all cause hospital readmissions, fewer subjects discharged to skilled nursing facilities, and fewer ER visits and doctor calls related to pain during the follow-up period.

To the objective of assessing the safety of preoperative dosing with 30 mg nanocrystalline meloxicam, the safety profile was observed to be consistent with that identified throughout the development program under postoperative dosing conditions. The types of treatment-emergent adverse events (TEAEs) continued to be consistent with the surgical population setting, as well as remaining primarily mild or moderate in intensity and generally not related to study treatment, with an overall lower incidence in the nanocrystalline meloxicam treatment group compared with placebo. Changes in laboratory assessments, vital signs, and surgical wound assessments were also comparable to placebo.

In sum, these results demonstrate the efficacy and safety of administering 30 mg nanocrystalline meloxicam preoperatively, as part of a multimodal regimen in subjects undergoing primary unilateral TKA.

### INCORPORATION BY REFERENCE

The disclosures of all publications, patents, patent applications and published patent applications referred to herein by an identifying citation are hereby incorporated herein by reference in their entirety.

In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A method of treating pain in a patient in need thereof, said patient being a patient who will be subjected to a surgical procedure, comprising administering meloxicam to the patient prior to start of the surgical procedure.

2. The method of claim 1, wherein the pain is an acute pain.

3. The method of claims 1 and 2, wherein meloxicam is present as nanocrystalline meloxicam.

4. The method of claim 3, wherein the nanocrystalline meloxicam is in a colloidal dispersion.

5. The method of claims 1-4, wherein meloxicam is administered to the patient in an amount ranging from about 5 mg to about 180 mg.

6. The method of claims 1-5, wherein meloxicam is administered to the patient in an amount of about 15 mg to about 60 mg.

7. The method of claims 1-6, wherein meloxicam is administered to the patient in an amount of about 30 mg.

8. The method of claims 1-7, wherein meloxicam is administered to the patient intravenously.

9. The method of claims 1-8, wherein meloxicam is administered to the patient intravenously over a course of about 5 seconds to about 60 seconds.

10. The method of claims 1-9, wherein meloxicam is administered to the patient intravenously over a course of about 10 seconds to about 60 seconds.

11. The method of claims 1-10, wherein meloxicam is administered to the patient intravenously over a course of about 15 seconds to about 30 seconds.

12. The method of claims 1-11, wherein meloxicam is administered to the patient intravenously over a course of about 15 seconds.

13. The method of claims 1-12, wherein meloxicam is administered to the patient within about 2 hours prior to start of the surgical procedure.

14. The method of claims 1-13, wherein meloxicam is administered to the patient within about 45 minutes prior to start of the surgical procedure.

15. The method of claims 1-14, wherein meloxicam is administered to the patient within about 30 minutes prior to start of the surgical procedure.

16. The method of claims 1-12, wherein meloxicam is administered to the patient prior to administration of anesthesia.

17. The method of claims 1-12, wherein meloxicam is administered to the patient after administration of anesthesia.

18. The method of claims 1-17, wherein the surgical procedure is performed on soft tissue, hard tissue or a combination thereof.

19. The method of claims 1-18, wherein the surgical procedure comprises an open surgical procedure.

20. The method of claims 1-18, wherein the surgical procedure comprises a laparoscopic surgical procedure.

21. The method of claims 1-20, wherein the surgical procedure comprises colorectal surgery.

22. The method of claims 1-20, wherein the surgical procedure comprises orthopedic surgery.

23. The method of claims 1-22, further comprising administering gabapentin, acetaminophen or a combination thereof to the patient prior to the start of the surgical procedure.

24. The method of claims 1-22, further comprising administering an analgesic to the patient before, during or after the surgical procedure.

25. The method of claim 24, wherein the analgesic comprises acetaminophen, opioid, or a combination thereof.

26. The method of claim 25, wherein the opioid is oxycodone.

27. The method of claims 1-26, further comprising administering meloxicam to the patient about every 18 hours to about every 26 hours after administering meloxicam prior to the start of the surgical procedure, and until the patient is no longer in need thereof.

28. The method of claim 27, comprising administering meloxicam to the patient about 24 hours, about 48 hours, about 72 hours and/or about 96 hours after administering meloxicam prior to start of the surgical procedure.

29. The method of claim 27, comprising administering meloxicam to the patient about every 24 hours after administering meloxicam prior to start of the surgical procedure.

30. A method of treating acute pain in a patient, said patient being a patient who will be subjected to soft tissue surgery, comprising administering NanoCrystal Colloidal Dispersion (NCD) meloxicam intravenously, over a course of about 15 seconds, to the patient in an amount of about 30 mg at about 30 minutes prior to start of soft tissue surgery.

31. The method of claim 30, further comprising administering NCD meloxicam to the patient about every 18 hours to about every 26 hours after administering NCD meloxicam prior to start of the surgery, and until the patient is no longer in need thereof.

32. The method of claim 31, further comprising administering NCD meloxicam to the patient about 24 hours, about 48 hours, and about 72 hours and/or about 96 hours after administering NCD meloxicam prior to start of the surgery.

33. A method of treating acute pain in a patient, said patient being a patient who will be subjected to hard tissue surgery, comprising administering NanoCrystal Colloidal Dispersion (NCD) meloxicam intravenously, over a course of about 15 seconds, to the patient in an amount of about 30 mg after the administration of anesthesia and prior to start of surgery.

34. The method of claim 33, further comprising administering NCD meloxicam to the patient about every 24 hours after administering NCD meloxicam prior to start of the surgery, until the patient is no longer in need thereof.

35. The method of claim 33, further comprising administering NCD meloxicam to the patient about 24 hours, about 48 hours, and about 72 hours and/or about 96 hours after administering NCD meloxicam prior to start of the surgery.

36. The method of claims 1-35, wherein the pain is a moderate to severe pain.

37. A method of treating pain in a patient in need thereof, comprising administering meloxicam intravenously to the patient in combination with acetaminophen and/or gabapentin.

38. The method of claim 37, wherein the pain is an acute pain.

39. The method of claims 37 and 38, wherein meloxicam is present as nanocrystalline meloxicam.

40. The method of claim 39, wherein the nanocrystalline meloxicam is in a colloidal dispersion.

41. The method of claims 37-40, wherein meloxicam is administered to the patient in an amount ranging from about 5 mg to about 180 mg.

42. The method of claims 37-41, wherein meloxicam is administered to the patient in an amount of about 15 mg to about 60 mg.

43. The method of claims 37-42, wherein meloxicam is administered to the patient in an amount of about 30 mg.

44. The method of claims 37-43, wherein meloxicam is administered to the patient intravenously over a course of about 5 seconds to 60 seconds.

45. The method of claims 37-44, wherein meloxicam is administered to the patient intravenously over a course of about 10 seconds to 60 seconds.

46. The method of claims 37-45, wherein meloxicam is administered to the patient intravenously over a course of about 15 seconds to 30 seconds.

47. The method of claims 37-46, wherein meloxicam is administered to the patient intravenously over a course of about 15 seconds.

48. The method of claims 37-47, comprising administering meloxicam to the patient in combination with acetaminophen.

49. The method of claim 48, wherein acetaminophen is administered orally, intravenously or a combination thereof.

50. The method of claims 48 and 49, wherein acetaminophen is administered in an amount of about 5 mg to about 1 g.

51. The method of claims 48-50, wherein acetaminophen is administered in an amount of about 200 mg to about 800 mg.

52. The method of claim 51, wherein acetaminophen is administered in an amount of about 650 mg.

53. The method of claims 37-52, comprising administering meloxicam to the patient in combination with gabapentin.

54. The method of claim 53, wherein gabapentin is administered orally.

55. The method of claims 53 and 54, wherein gabapentin is administered in an amount of about 200 mg to about 700 mg.

56. The method of claim 55, wherein gabapentin is administered in an amount of about 300 mg.

57. The method of claim 55, wherein gabapentin is administered in an amount of about 600 mg.

58. The method of claims 37-57, wherein meloxicam and acetaminophen and/or gabapentin are administered to the patient concurrently.

59. The method of claims 37-57, wherein meloxicam is administered to the patient within about 2 hours of acetaminophen and/or gabapentin administration.

60. The method of claims 37-57 and 59, wherein meloxicam is administered to the patient within about 1 hour of acetaminophen and/or gabapentin administration.

61. The method of claims 37-57, 59 and 60, wherein meloxicam is administered to the patient within about 30 minutes of acetaminophen and/or gabapentin administration.

62. The method of claims 37-61, wherein the patient is a patient who will be subjected to a surgical procedure and wherein meloxicam and acetaminophen and/or gabapentin are administered to the patient prior to start of the surgical procedure.

63. The method of claim 62, wherein acetaminophen and/or gabapentin are administered from about 30 to about 90 minutes prior to start of the surgical procedure.

64. The method of claim 62, wherein acetaminophen and/or gabapentin are administered before administration of anesthesia.

65. The method of claims 62-64, wherein meloxicam is administered after administration of anesthesia.

66. The method of claims 62-65, further comprising administering acetaminophen to the patient after completion of the surgical procedure.

67. The method of claims 62-66, wherein the surgical procedure is performed on soft tissue, hard tissue or a combination thereof.

68. The method of claims 62-67, wherein the surgical procedure comprises an open surgical procedure.

69. The method of claims 62-67, wherein the surgical procedure comprises a laparoscopic surgical procedure.

70. The method of claims 62-69, wherein the surgical procedure comprises colorectal surgery.

71. The method of claims 62-69, wherein the surgical procedure comprises unilateral total knee arthroplasty.

72. The method of claims 37-71, further comprising administering an antibiotic, an anti-nausea medication, a medication to treat or prevent excess bleeding, or a combination thereof, to the patient.

73. The method of claim 72, comprising administering the antibiotic, wherein the antibiotic is a prophylactic antibiotic.

74. The method of claims 72 and 73, comprising administering the anti-nausea medication, wherein the anti-nausea medication is ondansetron, dexamethasone, promethazine, scopolamine, or a combination thereof.

75. The method of claims 72-74, comprising administering the medication to treat or prevent excess bleeding, wherein the medication to treat or prevent excess bleeding is tranexamic acid.

76. A method of treating acute pain in a patient, said patient being a patient who will be subjected to hard tissue surgery, comprising administering NanoCrystal Colloidal Dispersion (NCD) meloxicam intravenously, over a course about 15 seconds, to the patient in an amount of about 30 mg, in combination with about 650 mg of acetaminophen and about 600 mg of gabapentin, wherein acetaminophen and gabapentin are administered at a time within a range of about 30-90 minutes prior to start of surgery, and wherein NCD meloxicam is administered about 30 minutes prior to start of surgery and after administration of anesthesia.

77. The method of claim 76, further comprising administering NCD meloxicam to the patient about every 18 hours to about every 26 hours after administering NCD meloxicam prior to the start of the surgery, and until the patient is no longer in need thereof.

78. The method of claim 76, further comprising administering NCD meloxicam to the patient about 24 hours, about 48 hours, about 72 hours and/or about 96 hours after administering NCD meloxicam prior to the start of the surgery.

79. A method of treating acute pain in a patient, said patient being a patient who will be subjected soft tissue surgery, comprising administering NanoCrystal Colloidal Dispersion (NCD) meloxicam intravenously, over a course of 15 seconds, to the patient in an amount of about 30 mg, in combination with about 650 mg of acetaminophen about 300 mg of gabapentin, wherein acetaminophen and gabapentin are administered at a time within a range of about 30-90 minutes prior to start of surgery, and wherein NCD meloxicam is administered about 30 minutes prior to start of surgery.

80. The method of claim 79, further comprising administering NCD meloxicam to the patient about every 18 hours to about every 26 hours after administering NCD meloxicam prior to the start of the, until the patient is no longer in need thereof.

81. The method of claim 79, further comprising administering NCD meloxicam to the patient about 24 hours, about 48 hours, about 72 hours and/or about 96 after administering NCD meloxicam prior to the start of the surgery.

82. The method of claims 62-65 and 67-81, wherein acetaminophen is administered to the patient prior to the start of the surgical procedure, and then about every 4-8 hours subsequent to the administration of acetaminophen prior to the start of the surgical procedure and until the patient is no longer in need thereof.

83. The method of claims 62-75, wherein meloxicam is administered about every 18 hours to about every 26 hours subsequent to the administration of meloxicam prior to start of the surgical procedure.

84. The method of 83, wherein meloxicam is administered about every 24 hours subsequent to the administration of meloxicam prior to start of the surgical procedure.

85. The method of claims 37-83, wherein the pain is a moderate to severe pain.

86. The method of claim 24, wherein the analgesic is administered concurrently with meloxicam prior to the start of surgery.

87. The method of claim 27-29, wherein the analgesic is administered concurrently with meloxicam.

88. The method of claims 33-35, further comprising administering an analgesic concurrently with meloxicam.
